(19) United States
(12) Patent Application Publication
(10) Pub. No.: US 2025/0361491 A1
(43) Pub. Date: Nov. 27, 2025

(54) METHODS OF PRODUCING AN ENVELOPED VIRUS

(71) Applicant: CSL Behring LLC, King of Prussia (PA)

(72) Inventors: Holger LAUX, Marburg (DE); Maximilian KLIMPEL, Marburg (DE); Nilakshi CHING, Victoria (AU)

(21) Appl. No.: 18/874,943

(22) PCT Filed: Jun. 15, 2023
(86) PCT No.: PCT/IB2023/056177

§ 371 Date: Dec. 13, 2024

Related U.S. Application Data
(60) Provisional application No. 63/366,408, filed on Jun. 15, 2022.
(60) Provisional application No. 63/498,041, filed on Apr. 25, 2023.

(51) Int. Cl. C12N 7/00; C12N 5/00; C12N 5/071; C12N 13/00; C12N 15/86

(52) U.S. Cl. CPC C12N 7/00; C12N 5/0018; C12N 5/0686; C12N 13/00; C12N 15/86; C12N 2523/00; C12N 2740/10043; C12N 2740/10052; C12N 2830/006

(57) ABSTRACT

The present disclosure relates to methods of producing an enveloped virus in a suspension cell culture, the method comprising culturing a suspension cell line expressing a tetracycline-suppressible gene expression system in a cell culture medium. The present disclosure also relates to stable producer cell clones capable of producing an enveloped virus in a suspension cell culture.

### RELATED APPLICATION DATA

The present application claims priority from United States Patent Application No. 63/366,408 entitled ‘Methods of producing an enveloped virus’ filed 15 Jun. 2022 and U.S. Patent Application No. 63/498,041 entitled ‘Methods of producing an enveloped virus’ filed 25 Apr. 2023. The entire contents of which are hereby incorporated by reference.

### FIELD

The present disclosure relates generally to the manufacturing of gene therapy products, and specifically to methods of producing an enveloped virus from a suspension cell culture expressing a tetracycline-suppressible gene expression system.

### BACKGROUND

Retroviruses, e.g., lentiviruses are one of the most studied viral vectors for gene therapy. Retroviruses in general are RNA-based viruses which integrate their genetic information into the target cell chromosomes permanently. The advantages of retroviruses include long-term transgene expression in target cells, a low immunogenic potential, and the ability to transduce into dividing and non-dividing cells.

Lentiviruses are genetically engineered and usually based on human immunodeficiency virus 1 (HIV-1). To increase safety, modem vectors contain only those HIV genes which are necessary for infection and gene delivery, but the genes necessary for replication and virulence factors have been removed. Often, the envelope protein of HIV-1 is exchanged with that of another virus to allow infection of a wide range of target cells, e.g., VSV-G protein from Vesicular stomatitis Indiana virus (VSV).

To produce lentiviruses, cells such as human embryonic kidney cells HEK 293T are transfected with 3-4 plasmids. These include the transfer plasmid with the gene of interest and several packaging plasmids encoding, vesicular stomatitis G protein (VSV-G), and essential viral proteins responsible for gene integration or self-assembly. These plasmids can be transiently transfected into the cells, or a producer cell line is created with stable integration of the plasmids with inducible promoters, in which lentivirus production can be induced.

Once the virus production has been induced, the release of the virus occurs by budding after successful assembly within the cells. The lentivirus is harvested from the producer cells and subsequently purified and concentrated in the downstream process.

Clinical-grade lentiviral vectors are most often produced by transient transfection of adherent cell lines. These production methods are cost intensive, require large amounts of GMP-grade plasmids and hamper process scalability and reproducibility.

Thus, there is a need in the art for an efficient process for producing lentiviruses in a cell culture system, e.g., for gene therapy.

### SUMMARY

In work leading up to the present invention, the inventors sought to produce a method for producing enveloped viruses, e.g., for gene therapy, at commercial scale and suitable for regulatory requirements.

The upstream process for producing an enveloped virus in a suspension cell culture expressing a tetracycline-suppressible (i.e., Tet-Off) gene expression system produced by the inventors includes a step to reduce the concentration of tetracycline or a derivative thereof (e.g., doxycycline) from the cell culture to induce viral production.

In adherent cell lines, media exchange is straightforward because the cells are grown on a surface, and media can be removed without disturbing the cells. In adherent cell lines, tetracycline or a derivative thereof can be removed by performing a media exchange to replace the tetracycline or derivative-containing cell culture medium with a tetracycline or derivative-free cell culture medium. In suspension cell lines, however, inducing viral production typically requires removing tetracycline or a derivative thereof from the cell culture medium by centrifugation to pellet the cells (i.e., removing the cells from suspension), followed by adding tetracycline or derivative-free cell culture medium, followed by resuspending the cells in the tetracycline or derivative-free cell culture medium by agitating the cells.

In developing these methods, the inventors determined that typical methods of reducing the concentration of tetracycline or a derivative thereof from the cell culture (e.g., centrifugation and resuspension) lowered the cell quality due to the high shear forces on the cells and also increased the risks of contamination due to open, manual steps.

To address this problem, the inventors identified that they could reduce the concentration of tetracycline or a derivative thereof in the cell culture by dilution or using an acoustic standing wave method. In one example, the inventors found that they could reduce the concentration of tetracycline or a derivative thereof in the cell culture by diluting the suspension cell culture with a tetracycline or derivative-free cell culture medium. In another example, the inventors found that they could reduce the concentration of tetracycline or a derivative thereof in the cell culture by retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

Thus, the findings by the inventors have provided methods of producing enveloped viruses.

In one example, the disclosure provides a method of producing an enveloped virus in a suspension cell culture, the method comprising culturing a suspension cell line expressing a tetracycline-suppressible gene expression system in a cell culture medium.

It will be apparent to the skilled person that the tetracycline-suppressible gene expression system is also known as a Tet-Off expression system.

In exemplary forms of the disclosure, the suspension cell line is a stable producer cell line, i.e., cells having stably incorporated therein the genetic material required to produce the lentivirus. Such cells are distinguished from cells having the genetic elements transiently incorporated therein.

An exemplary enveloped virus is a retrovirus. For example, the retrovirus is a lentivirus. For example, the lentivirus is HIV or a derivative thereof.

In one example, the suspension cell line is initially cultured in a cell culture medium comprising a sufficient amount of tetracycline or a derivative thereof to suppress production of the enveloped virus and allow expansion of the suspension cell line.

In one example, the initial cell culture is an expansion cell culture. For example, the initial cell culture is performed in an expansion (or N−1) bioreactor.

In one example, the sufficient amount of tetracycline or a derivative thereof in the cell culture medium is at least 0.1 ng/mL of cell culture medium. In one example, the sufficient amount of tetracycline or a derivative thereof in the cell culture medium is between about 0.1 ng/mL and about 10,000 ng/mL of cell culture medium. In one example, the sufficient amount of tetracycline or a derivative thereof in the cell culture medium is between about 0.1 ng/mL and about 1,000 ng/mL of cell culture medium. In one example, the sufficient amount of tetracycline or a derivative thereof in the cell culture medium is between about 0.1 ng/mL and about 100 ng/mL of cell culture medium. In one example, the sufficient amount of tetracycline or a derivative thereof in the cell culture medium is between about 0.1 ng/mL and about 10 ng/mL of cell culture medium. For example, the sufficient amount of tetracycline or a derivative thereof in the cell culture medium is about 0.1 ng/mL, or about 0.2 ng/mL, or about 0.3 ng/mL, or about 0.4 ng/mL, or about 0.5 ng/mL, or about 0.6 ng/mL, or about 0.7 ng/mL, or about 0.8 ng/mL, or about 0.9 ng/mL, or about 1 ng/mL of cell culture medium. In one example, the sufficient amount of tetracycline or a derivative thereof in the cell culture medium is at least 0.2 ng/mL. In one example, the sufficient amount of tetracycline or a derivative thereof in the cell culture medium is at least 0.5 ng/mL. In one example, the sufficient amount of tetracycline or a derivative thereof in the cell culture medium is between about 0.5 ng/mL and about 5 ng/mL, or about 1 ng/mL and about 5 ng/mL, or about 1.5 ng/mL and about 5 ng/mL, or about 2 ng/mL and about 5 ng/mL of cell culture medium. In one example, the sufficient amount of tetracycline or a derivative thereof in the cell culture medium is between 0.5 ng/mL and 5 ng/mL of cell culture medium. For example, the sufficient amount of tetracycline or a derivative thereof in the cell culture medium is 0.5 ng/mL, or ng/mL, or 1.5 ng/mL, or 2 ng/mL, or 2.5 ng/mL, or 3 ng/mL, or 3.5 ng/mL, or 4 ng/mL, or 4.5 ng/mL, or 5 ng/mL of cell culture medium. For example, the sufficient amount of tetracycline or a derivative thereof in the cell culture medium is 0.1 ng/mL of cell culture medium. In one example, the sufficient amount of tetracycline or a derivative thereof in the cell culture medium is 1 ng/mL of cell culture medium. In one example, the sufficient amount of tetracycline or a derivative thereof in the cell culture medium is 1.5 ng/mL of cell culture medium.

In one example, the method comprises reducing the concentration of tetracycline or a derivative thereof in the cell culture medium such that production of the enveloped virus is induced.

In one example, the concentration of tetracycline or derivative thereof is reduced by at least 50%, or at least 60%, or at least 70%, or at least 80%, or at least 85%, or at least 90%, or at least 95%. In one example, the concentration of tetracycline or derivative thereof is reduced by at least 50%. In one example, the concentration of tetracycline or derivative thereof is reduced by at least 60%. In one example, the concentration of tetracycline or derivative thereof is reduced by at least 70%. In one example, the concentration of tetracycline or derivative thereof is reduced by at least 80%. In one example, the concentration of tetracycline or derivative thereof is reduced by at least 85%. In one example, the concentration of tetracycline or derivative thereof is reduced by at least 90%. In one example, the concentration of tetracycline or derivative thereof is reduced by at least 95%.

In one example, the concentration of tetracycline or derivative thereof in the cell culture medium is reduced to a concentration of 0.5 ng/mL or less of cell culture medium. In one example, the concentration of tetracycline or derivative thereof in the cell culture medium is reduced to a concentration of 0.5 ng/mL to 0.001 ng/mL of cell culture medium. For example, the concentration of tetracycline or derivative thereof in the cell culture medium is reduced to a concentration of about 0.5 ng/mL, or about 0.45 ng/mL, or about 0.4 ng/mL, or about 0.35 ng/mL, or about 0.3 ng/mL, or about 0.25 ng/mL, or about 0.2 ng/mL, or about 0.1 ng/mL of cell culture medium. In one example, the concentration of tetracycline or derivative thereof in the cell culture medium is reduced to a concentration of 0.2 ng/mL or less of cell culture medium. In one example, the concentration of tetracycline or derivative thereof in the cell culture medium is reduced to a concentration of 0.1 ng/mL or less of cell culture medium. In one example, the concentration of tetracycline or derivative thereof in the cell culture medium is reduced to a concentration of 0.1 ng/mL to 0.001 ng/mL of cell culture medium. For example, the concentration of tetracycline or derivative thereof in the cell culture medium is reduced to a concentration of about 0.1 ng/mL, or about 0.05 ng/mL, or about 0.01 ng/mL, or about 0.005 ng/mL, or about 0.001 ng/mL of cell culture medium. In one example, the concentration of tetracycline or derivative thereof in the cell culture medium is reduced to a concentration of about 0.5 ng/mL of cell culture medium. In one example, the concentration of tetracycline or derivative thereof in the cell culture medium is reduced to a concentration of about 0.25 ng/mL of cell culture medium. In one example, the concentration of tetracycline or derivative thereof in the cell culture medium is reduced to a concentration of about 0.2 ng/mL of cell culture medium. In one example, the concentration of tetracycline or derivative thereof in the cell culture medium is reduced to a concentration of about 0.1 ng/mL of cell culture medium. In one example, the concentration of tetracycline or derivative thereof in the cell culture medium is reduced to a concentration of about 0.05 ng/mL of cell culture medium. In one example, the concentration of tetracycline or derivative thereof in the cell culture medium is reduced to a concentration of about 0.01 ng/mL of cell culture medium. In one example, the concentration of tetracycline or derivative thereof in the cell culture medium is reduced to a concentration of about 0.005 ng/mL of cell culture medium. In one example, the concentration of tetracycline or derivative thereof in the cell culture medium is reduced to a concentration of about 0.001 ng/mL of cell culture medium.

In one example the sufficient amount of tetracycline or a derivative thereof in the cell culture medium to suppress production of the enveloped virus and allow expansion of the suspension cell line is modulated prior to reducing the concentration of tetracycline or a derivative thereof in the cell culture medium such that production of the enveloped virus is induced. For example, the concentration of tetracycline or a derivative thereof in the expansion bioreactor is set to a first concentration for a first period of time, and is then modulated to a second concentration, which is lower than the first concentration, for a second period of time.

For example, the first concentration of tetracycline or a derivative thereof in the cell culture medium is at least 1 ng/mL of cell culture medium. In one example, the first concentration of tetracycline or a derivative thereof in the cell culture medium is between about 1 ng/mL and about 10 ng/mL of cell culture medium. For example, the first concentration of tetracycline or a derivative thereof in the cell culture medium is about 1 ng/mL, or about 2 ng/mL, or about 3 ng/mL, or about 4 ng/mL, or about 5 ng/mL, or about 6 ng/mL, or about 7 ng/mL, or about 8 ng/mL, or about 9 ng/mL, or about 10 ng/mL of cell culture medium. In one example, the first concentration of tetracycline or a derivative thereof in the cell culture medium is between about 1 ng/mL and about 5 ng/mL, or about 1.5 ng/mL and about 5 ng/mL, or about 2 ng/mL and about 5 ng/mL of cell culture medium, or about 2.5 ng/mL and about 5 ng/mL of cell culture medium. For example, the first concentration of tetracycline or a derivative thereof in the cell culture medium is 1 ng/mL, or 1.5 ng/mL, or 2 ng/mL, or 2.5 ng/mL, or 3 ng/mL, or 3.5 ng/mL, or 4 ng/mL, or 4.5 ng/mL, or 5 ng/mL of cell culture medium. For example, the first concentration of tetracycline or a derivative thereof in the cell culture medium is 2.5 ng/mL of cell culture medium.

For example, the concentration of tetracycline or a derivative thereof in the expansion bioreactor is set to the first concentration for a period of time of between about 1 and about 8 days. In one example, the first period of time is about 1 day, about 2 days, about 3 days, about 4 days, about 5 days, about 6 days, about 7 days, or about 8 days.

For example, the second concentration of tetracycline or a derivative thereof in the cell culture medium is at least 0.1 ng/mL of cell culture medium. In one example, the second concentration of tetracycline or a derivative thereof in the cell culture medium is between about 0.1 ng/mL and about 2.5 ng/mL of cell culture medium. For example, the second concentration of tetracycline or a derivative thereof in the cell culture medium is about 0.1 ng/mL, or about 0.2 ng/mL, or about 0.3 ng/mL, or about 0.4 ng/mL, or about 0.5 ng/mL, or about 0.6 ng/mL, or about 0.7 ng/mL, or about 0.8 ng/mL, or about 0.9 ng/mL, or about 1 ng/mL of cell culture medium. In one example, the second concentration of tetracycline or a derivative thereof in the cell culture medium is between about 0.1 ng/mL and about 2.5 ng/mL, or about 0.5 ng/mL and about 2.5 ng/mL, or about 1 ng/mL and about 2.5 ng/mL of cell culture medium, or about 1.5 ng/mL and about 2.5 ng/mL of cell culture medium. For example, the second concentration of tetracycline or a derivative thereof in the cell culture medium is 1 ng/mL, or 1.5 ng/mL, or 2 ng/mL, or 2.5 ng/mL of cell culture medium. For example, the second concentration of tetracycline or a derivative thereof in the cell culture medium is 1 ng/mL of cell culture medium.

For example, the concentration of tetracycline or a derivative thereof in the expansion bioreactor is set to the second concentration for a period of time of between about 1 and about 4 days. In one example, the first period of time is about 1 day, about 2 days, about 3 days, or about 4 days.

In one example, the concentration of tetracycline or a derivative thereof in the expansion bioreactor is set to a concentration of 2.5 ng/mL for about 2 days, and is then modulated to a concentration of 1.0 ng/mL for about 2 days.

In one example, the concentration of tetracycline or derivative thereof is reduced in the cell culture medium by:
(i) diluting the suspension cell culture with a tetracycline or derivative-free cell culture medium; or
(ii) retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

In one example, the concentration of tetracycline or derivative thereof is reduced in the cell culture medium by diluting the suspension cell culture with a tetracycline or derivative-free cell culture medium.

In one example, diluting the suspension cell culture comprises adding the tetracycline or derivative-free cell culture medium directly to the tetracycline or derivative-containing cell culture medium.

In one example, diluting the suspension cell culture comprises adding tetracycline or derivative-free cell culture medium to the suspension cell culture.

In one example, the suspension cell culture is diluted with the tetracycline or derivative-free cell culture medium at a ratio of between about 1:1 and 1:20. For example, the suspension cell culture is diluted with the tetracycline or derivative-free cell culture medium at a ratio of between 1:2 and 1:10, or a ratio of between 1:4 and 1:7. In one example, the suspension cell culture is diluted with the tetracycline or derivative-free cell culture medium at a ratio of between 1:2 and 1:10. For example, the suspension cell culture is diluted with the tetracycline or derivative-free cell culture medium at a ratio of about 1:2, or about 1:3, or about 1:4, or about 1:5, or about 1:6, or about 1:7, or about 1:8, or about 1:9, or about 1:10. In one example, the suspension cell culture is diluted with the tetracycline or derivative-free cell culture medium at a ratio of between 1:4 and 1:7. In one example, the suspension cell culture is diluted with the tetracycline or derivative-free cell culture medium at a ratio of about 1:4. In another example, the suspension cell culture is diluted with the tetracycline or derivative-free cell culture medium at a ratio of about 1:5. In a further example, the suspension cell culture is diluted with the tetracycline or derivative-free cell culture medium at a ratio of about 1:6. In one example, the suspension cell culture is diluted with the tetracycline or derivative-free cell culture medium at a ratio of about 1:7.

In one example, the concentration of tetracycline or derivative thereof is reduced in the cell culture medium by retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

In one example, the suspension cell line is initially seeded in the cell culture medium at a density of between about 1×10⁵ cells/mL and 1×10¹⁰ cells/mL of cell culture medium. For example, the suspension cell line is initially seeded in the cell culture medium at a density of between about 1×10⁵ cells/mL and 1×10¹⁰ cells/mL, or 1×10⁵ cells/mL and 1×10⁷ cells/mL, or about 0.1×10⁶ cells/mL and 1×10⁸ cells/mL, or about 0.5×10⁶ cells/mL and 1×10⁷ cells/mL, or about 0.5×10⁶ cells/mL and 5×10⁶ cells/mL, or about 0.5×10⁶ cells/mL and 2.5×10⁶ cells/mL of cell culture medium. In one example, the suspension cell line is initially seeded in the cell culture medium at a density of 1×10⁵ cells/mL and 1×10⁷ cells/m.L In one example, the suspension cell line is initially seeded in the cell culture medium at a density of 0.5×10⁶ cells/mL to 5.0×10⁶ cells/mL. In one example, the suspension cell line is initially seeded in the cell culture medium at a density of between 0.8×10⁶ cells/mL and 1.2×10⁶. In one example, the suspension cell line is initially seeded in the cell culture medium at a density of between 1×10⁶ cells/mL and 2.5×10⁶. In one example, the suspension cell line is initially seeded in the cell culture medium at a density of between 1.5×10⁶ cells/mL and 2×10⁶. In one example, the suspension cell line is initially seeded in the cell culture medium at a density of about 1×10⁵ cells/mL, or about 2×10⁵ cells/mL, or about 3×10⁵ cells/mL, or about 4×10⁵ cells/mL, or about 5×10⁵ cells/mL, or about 6×10⁵ cells/mL, or about 7×10⁵ cells/mL, or about 8×10⁵ cells/mL, or about 9×10⁵ cells/mL, or about 10×10⁵ cells/mL of cell culture medium. In one example, the suspension cell line is initially seeded in the cell culture medium at a density of about 1×10⁶ cells/mL, or about 2×10⁶ cells/mL, or about 3×10⁶ cells/mL, or about 4×10⁶ cells/mL, or about 5×10⁶ cells/mL, or about 6×10⁶ cells/mL, or about 7×10⁶ cells/mL, or about 8×10⁶ cells/mL, or about 9×10⁶ cells/mL, or about 10×10⁶ cells/mL of cell culture medium. In one example, the suspension cell line is initially seeded in the cell culture medium at a density of about 0.5×10⁶ cells/mL. In one example, the suspension cell line is initially seeded in the cell culture medium at a density of about 1×10⁶ cells/mL. In one example, the suspension cell line is initially seeded in the cell culture medium at a density of about 1.5×10⁶ cells/mL. In one example, the suspension cell line is initially seeded in the cell culture medium at a density of about 1.8×10⁶ cells/mL. In one example, the suspension cell line is initially seeded in the cell culture medium at a density of about 2×10⁶ cells/mL. In one example, the suspension cell line is initially seeded in the cell culture medium at a density of 2.5×10⁶ cells/mL. In one example, the suspension cell line is initially seeded in the cell culture medium at a density of 3.0×10⁶ cells/mL. In one example, the suspension cell line is initially seeded in the cell culture medium at a density of 3.5×10⁶ cells/mL. In one example, the suspension cell line is initially seeded in the cell culture medium at a density of 4.0×10⁶ cells/mL. In one example, the suspension cell line is initially seeded in the cell culture medium at a density of 4.5×10⁶ cells/mL. In one example, the suspension cell line is initially seeded in the cell culture medium at a density of 5.0×10⁶ cells/mL. In one example, the suspension cell line is initially seeded in the cell culture medium at a density of about 1×10⁷ cells/mL, or about 2×10⁷ cells/mL, or about 3×10⁷ cells/mL, or about 4×10⁷ cells/mL, or about 5×10⁷ cells/mL, or about 6×10⁷ cells/mL, or about 7×10⁷ cells/mL, or about 8×10⁷ cells/mL, or about 9×10⁷ cells/mL, or about 10×10⁷ cells/mL of cell culture medium. In one example, the suspension cell line is initially seeded in the cell culture medium at a density of about 1×10⁸ cells/mL, or about 2×10⁸ cells/mL, or about 3×10⁸ cells/mL, or about 4×10⁸ cells/mL, or about 5×10⁸ cells/mL, or about 6×10⁸ cells/mL, or about 7×10⁸ cells/mL, or about 8×10⁸ cells/mL, or about 9×10⁸ cells/mL, or about 10×10⁸ cells/mL of cell culture medium. In one example, the suspension cell line is initially seeded in the cell culture medium at a density of about 1×10⁹ cells/mL, or about 2×10⁹ cells/mL, or about 3×10⁹ cells/mL, or about 4×10⁹ cells/mL, or about 5×10⁹ cells/mL, or about 6×10⁹ cells/mL, or about 7×10⁹ cells/mL, or about 8×10⁹ cells/mL, or about 9×10⁹ cells/mL, or about 10×10⁹ cells/mL of cell culture medium.

In one example, the suspension cell line is grown to a viable cell density of between about 1×10⁵ cells/mL to about 1×10¹⁰ cells/mL prior to diluting the suspension cell culture with the tetracycline or derivative-free cell culture medium. For example, the suspension cell line is grown to a viable cell density of between about 1×10⁵ cells/mL and 1×10¹⁰ cells/mL, or about 0.1×10⁶ cells/mL and 1×10⁸ cells/mL, or about 0.5×10⁶ cells/mL and 1×10⁷ cells/mL, or about 0.5×10⁶ cells/mL and 5×10⁶ cells/mL, or about 0.5×10⁶ cells/mL and 2.5×10⁶ cells/mL of cell culture medium. In one example, the suspension cell line is grown to a viable cell density of between about 1×10⁶ cells/mL to about 1×10⁷ prior to diluting the suspension cell culture with the tetracycline or derivative-free cell culture medium. In another example, the suspension cell line is grown to a viable cell density of between about 6×10⁶ cells/mL to about 1×10⁷ prior to diluting the suspension cell culture with the tetracycline or derivative-free cell culture medium. In one example, the suspension cell line is grown to a viable cell density of between about 0.5×10⁶ cells/mL to 5.0×10⁶ cells/mL. In one example, the suspension cell line is grown to a viable cell density of between about 1×10⁶ cells/mL and 2.5×10⁶. In one example, the suspension cell line is grown to a viable cell density of between about 1.5×10⁶ cells/mL and 2×10⁶. In one example, the suspension cell line is grown to a viable cell density of about 1×10⁵ cells/mL, or about 2×10⁵ cells/mL, or about 3×10⁵ cells/mL, or about 4×10⁵ cells/mL, or about 5×10⁵ cells/mL, or about 6×10⁵ cells/mL, or about 7×10⁵ cells/mL, or about 8×10⁵ cells/mL, or about 9×10⁵ cells/mL, or about 10×10⁵ cells/mL of cell culture medium. In one example, the suspension cell line is grown to a viable cell density of about 1×10⁶ cells/mL, or about 2×10⁶ cells/mL, or about 3×10⁶ cells/mL, or about 4×10⁶ cells/mL, or about 5×10⁶ cells/mL, or about 6×10⁶ cells/mL, or about 7×10⁶ cells/mL, or about 8×10⁶ cells/mL, or about 9×10⁶ cells/mL, or about 10×10⁶ cells/mL of cell culture medium. In one example, the suspension cell line is grown to a viable cell density of about 0.5×10⁶ cells/mL. In one example, the suspension cell line is grown to a viable cell density of about 1×10⁶ cells/mL. In one example, the suspension cell line is grown to a viable cell density of about 1.5×10⁶ cells/mL. In one example, the suspension cell line is grown to a viable cell density of about 1.8×10⁶ cells/mL. In one example, the suspension cell line is grown to a viable cell density of about 2×10⁶ cells/mL. In one example, the suspension cell line is grown to a viable cell density of about 2.5×10⁶ cells/mL. In one example, the suspension cell line is grown to a viable cell density of about 3.0×10⁶ cells/mL. In one example, the suspension cell line is grown to a viable cell density of about 3.5×10⁶ cells/mL. In one example, the suspension cell line is grown to a viable cell density of about 4.0×10⁶ cells/mL. In one example, the suspension cell line is grown to a viable cell density of about 4.5×10⁶ cells/mL. In one example, the suspension cell line is grown to a viable cell density of about 5.0×10⁶ cells/mL. In one example, the suspension cell line is grown to a viable cell density of about 1×10⁷ cells/mL, or about 2×10⁷ cells/mL, or about 3×10⁷ cells/mL, or about 4×10⁷ cells/mL, or about 5×10⁷ cells/mL, or about 6×10⁷ cells/mL, or about 7×10⁷ cells/mL, or about 8×10⁷ cells/mL, or about 9×10⁷ cells/mL, or about 10×10⁷ cells/mL of cell culture medium. In one example, the suspension cell line is grown to a viable cell density of about 1×10⁸ cells/mL, or about 2×10⁸ cells/mL, or about 3×10⁸ cells/mL, or about 4×10⁸ cells/mL, or about 5×10⁸ cells/mL, or about 6×10⁸ cells/mL, or about 7×10⁸ cells/mL, or about 8×10⁸ cells/mL, or about 9×10⁸ cells/mL, or about 10×10⁸ cells/mL of cell culture medium. In one example, the suspension cell line is grown to a viable cell density of about 1×10⁹ cells/mL, or about 2×10⁹ cells/mL, or about 3×10⁹ cells/mL, or about 4×10⁹ cells/mL, or about 5×10⁹ cells/mL, or about 6×10⁹ cells/mL, or about 7×10⁹ cells/mL, or about 8×10⁹ cells/mL, or about 9×10⁹ cells/mL, or about 10×10⁹ cells/mL of cell culture medium.

In one example, the suspension cell line has a viability of at least 60% prior to diluting the suspension cell culture with the tetracycline or derivative-free cell culture medium. For example, the suspension cell line has a viability of at least 60%, or 70% or 80%, or 85%, or 90%, or 95%, or 99% prior to diluting the suspension cell culture with the tetracycline or derivative-free cell culture medium. In one example, the suspension cell line has a viability of at least 70% prior to diluting the suspension cell culture with the tetracycline or derivative-free cell culture medium. In one example, the suspension cell line has a viability of at least 80% prior to diluting the suspension cell culture with the tetracycline or derivative-free cell culture medium. In one example, the suspension cell line has a viability of at least 85% prior to diluting the suspension cell culture with the tetracycline or derivative-free cell culture medium. In one example, the suspension cell line has a viability of at least 90% prior to diluting the suspension cell culture with the tetracycline or derivative-free cell culture medium. In one example, the suspension cell line has a viability of at least 95% prior to diluting the suspension cell culture with the tetracycline or derivative-free cell culture medium. In one example, the suspension cell line has a viability of at least 99% prior to diluting the suspension cell culture with the tetracycline or derivative-free cell culture medium.

In one example, the suspension cell culture is operated in a batch, fed batch, continuous, semi-continuous, or perfusion mode. In one example, the cell culture is operated in batch mode. In another example, the cell culture is operated in fed batch mode. In a further example, the cell culture is operated in semi-continuous mode. In another example, the cell culture is operated in perfusion mode. In one example, the cell culture is operated in batch and perfusion mode. For example, the cell culture is initially operated in batch mode and subsequently operated in perfusion mode.

In one example of any method described herein, the method results in a viral infectious titer yield of at least 1×10⁵ transducing units (TU)/mL. For example, the method results in a viral infectious titer yield of between about 1×10⁵ TU/mL and 1×10¹⁰ TU/mL. In one example, the method results in a viral infectious titer yield of about 1×10⁵ TU/mL, or about 1.25×10⁵ TU/mL, or about 1.5×10⁵ TU/mL, or at least 1.75×10⁵ TU/mL, or at least 2×10⁵ TU/mL, or at least 2.5×10⁵ TU/mL, or at least 3×10⁵ TU/mL, or about 3.5×10⁵ TU/mL, or about 4×10⁵ TU/mL, or about 5×10⁵ TU/mL. In one example, the method results in a viral infectious titer yield of about 6×10⁵ TU/mL, or about 7×10⁵ TU/mL, or at least 8×10⁵ TU/mL, or at least 9×10⁵ TU/mL, or at least 10×10⁵ TU/mL. In another example, the method results in a viral infectious titer yield of about 1×10⁶ TU/mL, or about 1.5×10⁶ TU/mL, or about 2×10⁶ TU/mL, or about 5×10⁶ TU/mL, or about 7×10⁶ TU/mL, or about 10×10⁶ TU/mL. In a further example, the method results in a viral infectious titer yield of about 1×10⁷ TU/mL, or about 5×10⁷ TU/mL, or about 10×10⁸ TU/mL, or about 5×10⁸ TU/mL, or about 10×10⁸ TU/mL, or about 5×10⁹ TU/mL, or about 1×10¹⁰ TU/mL.

In one example, the method results in a viral infectious titer yield of at least 1×10⁵ transducing units (TU)/mL of culture medium at day 15 of culture. For example, the method results in a viral infectious titer yield of at least 1×10⁶ TU/mL of culture medium at day 15 of culture. For example, the method results in a viral infectious titer yield of about 1.5×10⁶ TU/mL, or about 2×10⁶ TU/mL, or about 5×10⁶ TU/mL, or about 7×10⁶ TU/mL, or about 10×10⁶ TU/mL of culture medium at day 15 of culture. In another example, the method results in a viral infectious titer yield of at least 1×10⁷ TU/mL of culture medium at day 15 of culture. For example, the method results in a viral infectious titer yield of about 1.1×10⁷ TU/mL, or about 1.2×10⁷ TU/mL, or about 1.3×10⁷ TU/mL, or about 1.4×10⁷ TU/mL, or about 1.5×10⁷ TU/mL of culture medium at day 15 of culture. In one example, the method results in a viral infectious titer yield of at least 1.5×10⁷ TU/mL of culture medium at day 15 of culture. For example, the method results in a viral infectious titer yield of about 1.6×10⁷ TU/mL, or about 1.7×10⁷ TU/mL, or about 1.8×10⁷ TU/mL, or about 1.9×10⁷ TU/mL of culture medium at day 15 of culture. In one example, the method results in a viral infectious titer yield of at least 2×10⁷ TU/mL of culture medium at day 15 of culture. For example, the method results in a viral infectious titer yield of about 2.1×10⁷ TU/mL, or about 2.2×10⁷ TU/mL, or about 2.3×10⁷ TU/mL, or about 2.4×10⁷ TU/mL, or about 2.5×10⁷ TU/mL, or about 2.6×10⁷ TU/mL, or about 2.7×10⁷ TU/mL, or about 2.8×10⁷ TU/mL, or about 2.9×10⁷ TU/mL of culture medium at day 15 of culture.

In one example, the method results in a viral infectious titer yield of at least 1×10⁵ transducing units (TU)/mL of culture medium at day 20 of culture. For example, the method results in a viral infectious titer yield of at least 1×10⁶ TU/mL of culture medium at day 20 of culture. For example, the method results in a viral infectious titer yield of about 1.5×10⁶ TU/mL, or about 2×10⁶ TU/mL, or about 5×10⁶ TU/mL, or about 7×10⁶ TU/mL, or about 10×10⁶ TU/mL of culture medium at day 20 of culture. In another example, the method results in a viral infectious titer yield of at least 1×10⁷ TU/mL of culture medium at day 20 of culture. For example, the method results in a viral infectious titer yield of about 1.1×10⁷ TU/mL, or about 1.2×10⁷ TU/mL, or about 1.3×10⁷ TU/mL, or about 1.4×10⁷ TU/mL, or about 1.5×10⁷ TU/mL of culture medium at day 20 of culture. In one example, the method results in a viral infectious titer yield of at least 1.5×10⁷ TU/mL of culture medium at day 20 of culture. For example, the method results in a viral infectious titer yield of about 1.6×10⁷ TU/mL, or about 1.7×10⁷ TU/mL, or about 1.8×10⁷ TU/mL, or about 1.9×10⁷ TU/mL of culture medium at day 20 of culture. In one example, the method results in a viral infectious titer yield of at least 2×10⁷ TU/mL of culture medium at day 20 of culture. For example, the method results in a viral infectious titer yield of about 2.1×10⁷ TU/mL, or about 2.2×10⁷ TU/mL, or about 2.3×10⁷ TU/mL, or about 2.4×10⁷ TU/mL, or about 2.5×10⁷ TU/mL, or about 2.6×10⁷ TU/mL, or about 2.7×10⁷ TU/mL, or about 2.8×10⁷ TU/mL, or about 2.9×10⁷ TU/mL of culture medium at day 20 of culture.

In one example, the method results in a viral infectious titer yield of at least 1×10⁵ transducing units (TU)/mL of culture medium at day 25 of culture. For example, the method results in a viral infectious titer yield of at least 1×10⁶ TU/mL of culture medium at day 25 of culture. For example, the method results in a viral infectious titer yield of about 1.5×10⁶ TU/mL, or about 2×10⁶ TU/mL, or about 5×10⁶ TU/mL, or about 7×10⁶ TU/mL, or about 10×10⁶ TU/mL of culture medium at day 25 of culture. In another example, the method results in a viral infectious titer yield of at least 1×10⁷ TU/mL of culture medium at day 25 of culture. For example, the method results in a viral infectious titer yield of about 1.1×10⁷ TU/mL, or about 1.2×10⁷ TU/mL, or about 1.3×10⁷ TU/mL, or about 1.4×10⁷ TU/mL, or about 1.5×10⁷ TU/mL of culture medium at day 25 of culture. In one example, the method results in a viral infectious titer yield of at least 1.5×10⁷ TU/mL of culture medium at day 25 of culture. For example, the method results in a viral infectious titer yield of about 1.6×10⁷ TU/mL, or about 1.7×10⁷ TU/mL, or about 1.8×10⁷ TU/mL, or about 1.9×10⁷ TU/mL of culture medium at day 25 of culture. In one example, the method results in a viral infectious titer yield of at least 2×10⁷ TU/mL of culture medium at day 25 of culture. For example, the method results in a viral infectious titer yield of about 2.1×10⁷ TU/mL, or about 2.2×10⁷ TU/mL, or about 2.3×10⁷ TU/mL, or about 2.4×10⁷ TU/mL, or about 2.5×10⁷ TU/mL, or about 2.6×10⁷ TU/mL, or about 2.7×10⁷ TU/mL, or about 2.8×10⁷ TU/mL, or about 2.9×10⁷ TU/mL of culture medium at day 25 of culture.

In one example, the method results in a viral infectious titer yield of at least 1×10⁵ transducing units (TU)/mL of culture medium at day 30 of culture. For example, the method results in a viral infectious titer yield of at least 1×10⁶ TU/mL of culture medium at day 30 of culture. For example, the method results in a viral infectious titer yield of about 1.5×10⁶ TU/mL, or about 2×10⁶ TU/mL, or about 5×10⁶ TU/mL, or about 7×10⁶ TU/mL, or about 10×10⁶ TU/mL of culture medium at day 30 of culture. In another example, the method results in a viral infectious titer yield of at least 1×10⁷ TU/mL of culture medium at day 30 of culture. For example, the method results in a viral infectious titer yield of about 1.1×10⁷ TU/mL, or about 1.2×10⁷ TU/mL, or about 1.3×10⁷ TU/mL, or about 1.4×10⁷ TU/mL, or about 1.5×10⁷ TU/mL of culture medium at day 30 of culture. In one example, the method results in a viral infectious titer yield of at least 1.5×10⁷ TU/mL of culture medium at day 30 of culture. For example, the method results in a viral infectious titer yield of about 1.6×10⁷ TU/mL, or about 1.7×10⁷ TU/mL, or about 1.8×10⁷ TU/mL, or about 1.9×10⁷ TU/mL of culture medium at day 30 of culture. In one example, the method results in a viral infectious titer yield of at least 2×10⁷ TU/mL of culture medium at day 30 of culture. For example, the method results in a viral infectious titer yield of about 2.1×10⁷ TU/mL, or about 2.2×10⁷ TU/mL, or about 2.3×10⁷ TU/mL, or about 2.4×10⁷ TU/mL, or about 2.5×10⁷ TU/mL, or about 2.6×10⁷ TU/mL, or about 2.7×10⁷ TU/mL, or about 2.8×10⁷ TU/mL, or about 2.9×10⁷ TU/mL of culture medium at day 30 of culture.

In one example, the method results in a viral infectious titer yield of at least 1×10⁵ transducing units (TU)/mL of culture medium at day 35 of culture. For example, the method results in a viral infectious titer yield of at least 1×10⁶ TU/mL of culture medium at day 35 of culture. For example, the method results in a viral infectious titer yield of about 1.5×10⁶ TU/mL, or about 2×10⁶ TU/mL, or about 5×10⁶ TU/mL, or about 7×10⁶ TU/mL, or about 10×10⁶ TU/mL of culture medium at day 35 of culture. In another example, the method results in a viral infectious titer yield of at least 1×10⁷ TU/mL of culture medium at day 35 of culture. For example, the method results in a viral infectious titer yield of about 1.1×10⁷ TU/mL, or about 1.2×10⁷ TU/mL, or about 1.3×10⁷ TU/mL, or about 1.4×10⁷ TU/mL, or about 1.5×10⁷ TU/mL of culture medium at day 35 of culture. In one example, the method results in a viral infectious titer yield of at least 1.5×10⁷ TU/mL of culture medium at day 35 of culture. For example, the method results in a viral infectious titer yield of about 1.6×10⁷ TU/mL, or about 1.7×10⁷ TU/mL, or about 1.8×10⁷ TU/mL, or about 1.9×10⁷ TU/mL of culture medium at day 35 of culture. In one example, the method results in a viral infectious titer yield of at least 2×10⁷ TU/mL of culture medium at day 35 of culture. For example, the method results in a viral infectious titer yield of about 2.1×10⁷ TU/mL, or about 2.2×10⁷ TU/mL, or about 2.3×10⁷ TU/mL, or about 2.4×10⁷ TU/mL, or about 2.5×10⁷ TU/mL, or about 2.6×10⁷ TU/mL, or about 2.7×10⁷ TU/mL, or about 2.8×10⁷ TU/mL, or about 2.9×10⁷ TU/mL of culture medium at day 35 of culture.

In one example, the method results in a viable cell density of at least about 1×10⁵ cells/mL of culture medium at day 15 of culture. For example, the method results in a viable cell density of at least about 1×10⁶ cells/mL of culture medium at day 15 of culture. In one example, the method results in a viable cell density of at least about 1.5×10⁶ cells/mL, or about 2×10⁶ cells/mL, or about 5×10⁶ cells/mL, or about 7×10⁶ cells/mL, or about 10×10⁶ cells/mL of culture medium at day 15 of culture. In one example, the method results in a viable cell density of at least about 1×10⁷ cells/mL of culture medium at day 15 of culture. In one example, the method results in a viable cell density of at least about 1.1×10⁷ cells/mL, or about 1.2×10⁷ cells/mL, or about 1.3×10⁷ cells/mL, or about 1.4×10⁷ cells/mL, or about 1.5×10⁷ cells/mL of culture medium at day 15 of culture. In one example, the method results in a viable cell density of at least about 1.5×10⁷ cells/mL of culture medium at day 15 of culture. In one example, the method results in a viable cell density of at least about 1.5×10⁷ cells/mL, or about 1.6×10⁷ cells/mL, or about 1.7×10⁷ cells/mL, or about 1.8×10⁷ cells/mL, or about 1.9×10⁷ cells/mL, or about 2.0×10⁷ cells/mL of culture medium at day 15 of culture. In one example, the method results in a viable cell density of at least about 2.0×10⁷ cells/mL of culture medium at day 15 of culture. In one example, the method results in a viable cell density of at least about 2.1×10⁷ cells/mL, or about 2.2×10⁷ cells/mL, or about 2.3×10⁷ cells/mL, or about 2.4×10⁷ cells/mL, or about 2.5×10⁷ cells/mL of culture medium at day 15 of culture. In one example, the method results in a viable cell density of at least about 2.5×10⁷ cells/mL of culture medium at day 15 of culture. In one example, the method results in a viable cell density of at least about 2.6×10⁷ cells/mL, or about 2.7×10⁷ cells/mL, or about 2.8×10⁷ cells/mL, or about 2.9×10⁷ cells/mL, or about 3.0×10⁷ cells/mL of culture medium at day 15 of culture.

In one example, the method results in a viable cell density of at least about 1×10⁵ cells/mL of culture medium at day 20 of culture. For example, the method results in a viable cell density of at least about 1×10⁶ cells/mL of culture medium at day 20 of culture. In one example, the method results in a viable cell density of at least about 1.5×10⁶ cells/mL, or about 2×10⁶ cells/mL, or about 5×10⁶ cells/mL, or about 7×10⁶ cells/mL, or about 10×10⁶ cells/mL of culture medium at day 20 of culture. In one example, the method results in a viable cell density of at least about 1×10⁷ cells/mL of culture medium at day 20 of culture. In one example, the method results in a viable cell density of at least about 1.1×10⁷ cells/mL, or about 1.2×10⁷ cells/mL, or about 1.3×10⁷ cells/mL, or about 1.4×10⁷ cells/mL, or about 1.5×10⁷ cells/mL of culture medium at day 20 of culture. In one example, the method results in a viable cell density of at least about 1.5×10⁷ cells/mL of culture medium at day 20 of culture. In one example, the method results in a viable cell density of at least about 1.5×10⁷ cells/mL, or about 1.6×10⁷ cells/mL, or about 1.7×10⁷ cells/mL, or about 1.8×10⁷ cells/mL, or about 1.9×10⁷ cells/mL, or about 2.0×10⁷ cells/mL of culture medium at day 20 of culture. In one example, the method results in a viable cell density of at least about 2.0×10⁷ cells/mL of culture medium at day 20 of culture. In one example, the method results in a viable cell density of at least about 2.1×10⁷ cells/mL, or about 2.2×10⁷ cells/mL, or about 2.3×10⁷ cells/mL, or about 2.4×10⁷ cells/mL, or about 2.5×10⁷ cells/mL of culture medium at day 20 of culture. In one example, the method results in a viable cell density of at least about 2.5×10⁷ cells/mL of culture medium at day 20 of culture. In one example, the method results in a viable cell density of at least about 2.6×10⁷ cells/mL, or about 2.7×10⁷ cells/mL, or about 2.8×10⁷ cells/mL, or about 2.9×10⁷ cells/mL, or about 3.0×10⁷ cells/mL of culture medium at day 20 of culture.

In one example, the method results in a viable cell density of at least about 1×10⁵ cells/mL of culture medium at day 25 of culture. For example, the method results in a viable cell density of at least about 1×10⁶ cells/mL of culture medium at day 25 of culture. In one example, the method results in a viable cell density of at least about 1.5×10⁶ cells/mL, or about 2×10⁶ cells/mL, or about 5×10⁶ cells/mL, or about 7×10⁶ cells/mL, or about 10×10⁶ cells/mL of culture medium at day 25 of culture. In one example, the method results in a viable cell density of at least about 1×10⁷ cells/mL of culture medium at day 25 of culture. In one example, the method results in a viable cell density of at least about 1.1×10⁷ cells/mL, or about 1.2×10⁷ cells/mL, or about 1.3×10⁷ cells/mL, or about 1.4×10⁷ cells/mL, or about 1.5×10⁷ cells/mL of culture medium at day 25 of culture. In one example, the method results in a viable cell density of at least about 1.5×10⁷ cells/mL of culture medium at day 25 of culture. In one example, the method results in a viable cell density of at least about 1.5×10⁷ cells/mL, or about 1.6×10⁷ cells/mL, or about 1.7×10⁷ cells/mL, or about 1.8×10⁷ cells/mL, or about 1.9×10⁷ cells/mL, or about 2.0×10⁷ cells/mL of culture medium at day 25 of culture. In one example, the method results in a viable cell density of at least about 2.0×10⁷ cells/mL of culture medium at day 25 of culture. In one example, the method results in a viable cell density of at least about 2.1×10⁷ cells/mL, or about 2.2×10⁷ cells/mL, or about 2.3×10⁷ cells/mL, or about 2.4×10⁷ cells/mL, or about 2.5×10⁷ cells/mL of culture medium at day 25 of culture. In one example, the method results in a viable cell density of at least about 2.5×10⁷ cells/mL of culture medium at day 25 of culture. In one example, the method results in a viable cell density of at least about 2.6×10⁷ cells/mL, or about 2.7×10⁷ cells/mL, or about 2.8×10⁷ cells/mL, or about 2.9×10⁷ cells/mL, or about 3.0×10⁷ cells/mL of culture medium at day 25 of culture.

In one example, the method results in a viable cell density of at least about 1×10⁵ cells/mL of culture medium at day 30 of culture. For example, the method results in a viable cell density of at least about 1×10⁶ cells/mL of culture medium at day 30 of culture. In one example, the method results in a viable cell density of at least about 1.5×10⁶ cells/mL, or about 2×10⁶ cells/mL, or about 5×10⁶ cells/mL, or about 7×10⁶ cells/mL, or about 10×10⁶ cells/mL of culture medium at day 30 of culture. In one example, the method results in a viable cell density of at least about 1×10⁷ cells/mL of culture medium at day 30 of culture. In one example, the method results in a viable cell density of at least about 1.1×10⁷ cells/mL, or about 1.2×10⁷ cells/mL, or about 1.3×10⁷ cells/mL, or about 1.4×10⁷ cells/mL, or about 1.5×10⁷ cells/mL of culture medium at day 30 of culture. In one example, the method results in a viable cell density of at least about 1.5×10⁷ cells/mL of culture medium at day 30 of culture. In one example, the method results in a viable cell density of at least about 1.5×10⁷ cells/mL, or about 1.6×10⁷ cells/mL, or about 1.7×10⁷ cells/mL, or about 1.8×10⁷ cells/mL, or about 1.9×10⁷ cells/mL, or about 2.0×10⁷ cells/mL of culture medium at day 30 of culture. In one example, the method results in a viable cell density of at least about 2.0×10⁷ cells/mL of culture medium at day 30 of culture. In one example, the method results in a viable cell density of at least about 2.1×10⁷ cells/mL, or about 2.2×10⁷ cells/mL, or about 2.3×10⁷ cells/mL, or about 2.4×10⁷ cells/mL, or about 2.5×10⁷ cells/mL of culture medium at day 30 of culture. In one example, the method results in a viable cell density of at least about 2.5×10⁷ cells/mL of culture medium at day 30 of culture. In one example, the method results in a viable cell density of at least about 2.6×10⁷ cells/mL, or about 2.7×10⁷ cells/mL, or about 2.8×10⁷ cells/mL, or about 2.9×10⁷ cells/mL, or about 3.0×10⁷ cells/mL of culture medium at day 30 of culture.

In one example, the method results in a viable cell density of at least about 1×10⁵ cells/mL of culture medium at day 35 of culture. For example, the method results in a viable cell density of at least about 1×10⁶ cells/mL of culture medium at day 35 of culture. In one example, the method results in a viable cell density of at least about 1.5×10⁶ cells/mL, or about 2×10⁶ cells/mL, or about 5×10⁶ cells/mL, or about 7×10⁶ cells/mL, or about 10×10⁶ cells/mL of culture medium at day 35 of culture. In one example, the method results in a viable cell density of at least about 1×10⁷ cells/mL of culture medium at day 35 of culture. In one example, the method results in a viable cell density of at least about 1.1×10⁷ cells/mL, or about 1.2×10⁷ cells/mL, or about 1.3×10⁷ cells/mL, or about 1.4×10⁷ cells/mL, or about 1.5×10⁷ cells/mL of culture medium at day 35 of culture. In one example, the method results in a viable cell density of at least about 1.5×10⁷ cells/mL of culture medium at day 35 of culture. In one example, the method results in a viable cell density of at least about 1.5×10⁷ cells/mL, or about 1.6×10⁷ cells/mL, or about 1.7×10⁷ cells/mL, or about 1.8×10⁷ cells/mL, or about 1.9×10⁷ cells/mL, or about 2.0×10⁷ cells/mL of culture medium at day 35 of culture. In one example, the method results in a viable cell density of at least about 2.0×10⁷ cells/mL of culture medium at day 35 of culture. In one example, the method results in a viable cell density of at least about 2.1×10⁷ cells/mL, or about 2.2×10⁷ cells/mL, or about 2.3×10⁷ cells/mL, or about 2.4×10⁷ cells/mL, or about 2.5×10⁷ cells/mL of culture medium at day 35 of culture. In one example, the method results in a viable cell density of at least about 2.5×10⁷ cells/mL of culture medium at day 35 of culture. In one example, the method results in a viable cell density of at least about 2.6×10⁷ cells/mL, or about 2.7×10⁷ cells/mL, or about 2.8×10⁷ cells/mL, or about 2.9×10⁷ cells/mL, or about 3.0×10⁷ cells/mL of culture medium at day 35 of culture.

In one example, the cell line has a viability of at least 70% at day 15 of culture. For example, the cell line has a viability of about 70%, or about 75% or about 80% at day 15 of culture. In one example, the cell line has a viability of at least 75% at day 15 of culture. In another example, the cell line has a viability of at least 80% at day 15 of culture. For example, the cell line has a viability of about 80% or about 85% or about 90% at day 15 of culture. In one example, the cell line has a viability of about 80% at day 15 of culture. For example, a viability of about 81%, or about 82%, or about 83%, or about 84%. In another example, the cell line has a viability of about 85% at day 15 of culture. For example, a viability of about 86%, or about 87%, or about 88%, or about 89% at day 15 of culture. In a further example, the cell line has a viability of about 90% at day 15 of culture. In one example, the cell line has a viability of at least 90% at day 15 of culture. For example, the cell line has a viability of about 90%, or about 91%, or about 92%, or about 93%, or about 94%, or about 95% at day 15 of culture. In one example, the cell line has a viability of at least 95% at day 15 of culture. For example, the cell line has a viability of about 96%, or about 97%, or about 98%, or about 99% at day 15 of culture.

In one example, the cell line has a viability of at least 70% at day 20 of culture. For example, the cell line has a viability of about 70%, or about 75% or about 80% at day 20 of culture. In one example, the cell line has a viability of at least 75% at day 20 of culture. In another example, the cell line has a viability of at least 80% at day 20 of culture. For example, the cell line has a viability of about 80% or about 85% or about 90% at day 20 of culture. In one example, the cell line has a viability of about 80% at day 20 of culture. For example, a viability of about 81%, or about 82%, or about 83%, or about 84%. In another example, the cell line has a viability of about 85% at day 20 of culture. For example, a viability of about 86%, or about 87%, or about 88%, or about 89% at day 20 of culture. In a further example, the cell line has a viability of about 90% at day 20 of culture. In one example, the cell line has a viability of at least 90% at day 20 of culture. For example, the cell line has a viability of about 90%, or about 91%, or about 92%, or about 93%, or about 94%, or about 95% at day 20 of culture. In one example, the cell line has a viability of at least 95% at day 20 of culture. For example, the cell line has a viability of about 96%, or about 97%, or about 98%, or about 99% at day 20 of culture.

In one example, the cell line has a viability of at least 70% at day 25 of culture. For example, the cell line has a viability of about 70%, or about 75% or about 80% at day 25 of culture. In one example, the cell line has a viability of at least 75% at day 25 of culture. In another example, the cell line has a viability of at least 80% at day 25 of culture. For example, the cell line has a viability of about 80% or about 85% or about 90% at day 25 of culture. In one example, the cell line has a viability of about 80% at day 25 of culture. For example, a viability of about 81%, or about 82%, or about 83%, or about 84%. In another example, the cell line has a viability of about 85% at day 25 of culture. For example, a viability of about 86%, or about 87%, or about 88%, or about 89% at day 25 of culture. In a further example, the cell line has a viability of about 90% at day 25 of culture. In one example, the cell line has a viability of at least 90% at day 25 of culture. For example, the cell line has a viability of about 90%, or about 91%, or about 92%, or about 93%, or about 94%, or about 95% at day 25 of culture. In one example, the cell line has a viability of at least 95% at day 25 of culture. For example, the cell line has a viability of about 96%, or about 97%, or about 98%, or about 99% at day 25 of culture.

In one example, the cell line has a viability of at least 70% at day 30 of culture. For example, the cell line has a viability of about 70%, or about 75% or about 80% at day 30 of culture. In one example, the cell line has a viability of at least 75% at day 30 of culture. In another example, the cell line has a viability of at least 80% at day 30 of culture. For example, the cell line has a viability of about 80% or about 85% or about 90% at day 30 of culture. In one example, the cell line has a viability of about 80% at day 30 of culture. For example, a viability of about 81%, or about 82%, or about 83%, or about 84%. In another example, the cell line has a viability of about 85% at day 30 of culture. For example, a viability of about 86%, or about 87%, or about 88%, or about 89% at day 30 of culture. In a further example, the cell line has a viability of about 90% at day 30 of culture. In one example, the cell line has a viability of at least 90% at day 30 of culture. For example, the cell line has a viability of about 90%, or about 91%, or about 92%, or about 93%, or about 94%, or about 95% at day 30 of culture. In one example, the cell line has a viability of at least 95% at day 30 of culture. For example, the cell line has a viability of about 96%, or about 97%, or about 98%, or about 99% at day 30 of culture.

In one example, the cell line has a viability of at least 70% at day 35 of culture. For example, the cell line has a viability of about 70%, or about 75% or about 80% at day 35 of culture. In one example, the cell line has a viability of at least 75% at day 35 of culture. In another example, the cell line has a viability of at least 80% at day 35 of culture. For example, the cell line has a viability of about 80% or about 85% or about 90% at day 35 of culture. In one example, the cell line has a viability of about 80% at day 35 of culture. For example, a viability of about 81%, or about 82%, or about 83%, or about 84%. In another example, the cell line has a viability of about 85% at day 35 of culture. For example, a viability of about 86%, or about 87%, or about 88%, or about 89% at day 35 of culture. In a further example, the cell line has a viability of about 90% at day 35 of culture. In one example, the cell line has a viability of at least 90% at day 35 of culture. For example, the cell line has a viability of about 90%, or about 91%, or about 92%, or about 93%, or about 94%, or about 95% at day 35 of culture. In one example, the cell line has a viability of at least 95% at day 35 of culture. For example, the cell line has a viability of about 96%, or about 97%, or about 98%, or about 99% at day 35 of culture.

In one example, the method increases the virus infectious titer yield by at least 2% or 3% or 4% or 5% or 10% or 15% or 20%. In one example, the method increases the virus infectious titer yield by at least 10%.

In one example, the tetracycline derivative is selected from the group consisting of minocycline, doxycycline, demeclocycline, oxytetracycline, and tigecycline. In one example, the tetracycline derivative is doxycycline.

In one example, the suspension cell culture has a volume of greater than about 1 L, about 2 L, about 5 L, about 10 L, about 50 L, about 100 L, about 500 L, about 1000 L, about 5,000 L, about 10,000 L, or about 15,000 L. For example, the suspension cell culture has a volume of between about 1 L and 1000 L. In one example, the suspension cell culture has a volume of about 1 L. In another example, the suspension cell culture has a volume of about 5 L. In a further example, the suspension cell culture has a volume of about 10 L. In one example, the suspension cell culture has a volume of about 50 L. In another example, the suspension cell culture has a volume of about 100 L. In a further example, the suspension cell culture has a volume of about 500 L. In one example, the suspension cell culture has a volume of about 1000 L. In another example, the suspension cell culture has a volume of about 5000 L. In another example, the suspension cell culture has a volume of about 10,000 L. In another example, the suspension cell culture has a volume of about 15,000 L.

In one example, the suspension cell culture is operated with a dissolved carbon dioxide (CO₂) level of between 4% and 8%. For example, the suspension cell culture is operated with about 4% CO₂. In another example, the suspension cell culture is operated with about 5% CO₂. In a further example, the suspension cell culture is operated with about 6% CO₂. In a further example, the suspension cell culture is operated with about 7% CO₂. In a further example, the suspension cell culture is operated with about 8% CO₂.

In one example, the suspension cell culture is operated at a pH of between 6.0 and 8.0. In one example, the suspension cell culture is at a pH of between about 6.5 and 7.5. For example, the pH is between about 6.90 and about 7.3. In one example, the pH is about 7.1. In one example, the pH is about 6.5. In one example, the pH is about 6.6. In one example, the pH is about 6.7. In one example, the pH is about 6.8. In one example, the pH is about 6.9. In one example, the pH is about 7.0. In one example, the pH is about 7.1. In one example, the pH is about 7.2. In one example, the pH is about 7.3. In one example, the pH is about 7.4. In one example, the pH is about 7.5.

In one example, the suspension cell culture is operated at a temperature of between about 35° C. and 39° C. For example, the suspension cell culture is at a temperature of about 35° C., or about 35.5° C., or about 36° C., or about 36.5° C., or about 37° C., or about 37.5° C., or about 38° C., or about 38.5° C., or about 39° C. In one example, the suspension cell culture is at a temperature of between about 36.5° C. and about 37.5° C. For example, the suspension cell culture is at a temperature of about 37.0° C. In one example, the suspension cell culture is at a temperature of between about 38° C. and about 39° C. For example, the suspension cell culture is at a temperature of about 38.5° C.

In one example, the suspension cell culture is at a pH of between 6.0 and 8.0 and/or at a temperature of between 35-39° C. In one example, the suspension cell culture is at a pH of between 6.0 and 8.0 and/or at a temperature of between 37-38.5° C. For example, the suspension cell culture is at a pH of between about 6.8 and about 7.1 and/or at a temperature of between about 37° C. and about 38.5° C. In one example, the suspension cell culture is at a pH of between about 6.8 and about 7.1 and at a temperature of between about 37° C. and about 38.5° C. In one example, the suspension cell culture is at a pH of about 6.9 to about 7.0 and at a temperature of about 37.0° C.

In one example, the method further comprises selecting a cell clone capable of producing the enveloped virus at a high infectious titer and viability for at least 15 days. For example, the method further comprises selecting a cell clone capable of producing the enveloped virus at an infectious titer of at least 5×10⁶ TU/mL of culture medium and/or a viability of at least 75% for at least 15 days and/or a viable cell density of at least 1×10⁷ cells/mL of culture medium.

In one example, the method further comprises selecting a cell clone capable of producing the enveloped virus at a high infectious titer and viability for at least 20 days. For example, the method further comprises selecting a cell clone capable of producing the enveloped virus at an infectious titer of at least 5×10⁶ TU/mL of culture medium and/or a viability of at least 75% for at least 20 days and/or a viable cell density of at least 1×10⁷ cells/mL of culture medium.

In one example, the method further comprises selecting a cell clone capable of producing the enveloped virus at a high infectious titer and viability for at least 25 days. For example, the method further comprises selecting a cell clone capable of producing the enveloped virus at an infectious titer of at least 5×10⁶ TU/mL of culture medium and/or a viability of at least 75% for at least 25 days and/or a viable cell density of at least 1×10⁷ cells/mL of culture medium.

In one example, the method further comprises selecting a cell clone capable of producing the enveloped virus at a high infectious titer and viability for at least 30 days. For example, the method further comprises selecting a cell clone capable of producing the enveloped virus at an infectious titer of at least 5×10⁶ TU/mL of culture medium and/or a viability of at least 75% for at least 30 days and/or a viable cell density of at least 1×10⁷ cells/mL of culture medium.

In one example, the method further comprises selecting a cell clone capable of producing the enveloped virus at a high infectious titer and viability for at least 35 days. For example, the method further comprises selecting a cell clone capable of producing the enveloped virus at an infectious titer of at least 5×10⁶ TU/mL of culture medium and/or a viability of at least 75% for at least 35 days and/or a viable cell density of at least 1×10⁷ cells/mL of culture medium.

Additional methods of selecting a suitable cell clone will be apparent to the skilled person and/or are described herein and include for example, stability and/or mutation status of the cell clone and ability of the purified virus to transduce haematopoietic stem cells.

In one example, the method further comprises purifying the enveloped virus from the suspension cell culture.

Methods of purifying the enveloped virus from the suspension cell culture will be apparent to the skilled person and/or are described herein. In one example, purifying the enveloped virus comprises one or more steps selected from the group consisting of clarification filtration, anion exchange chromatography, concentration and diafiltration.

In one example, a method of the disclosure additionally comprises performing sterile filtration. For example, the sterile filtration is performed prior to concentrating and diafiltering the eluted virus. In an alternative example, the sterile filtration is performed after concentrating and diafiltering the eluted virus.

In one example, the method additionally comprising formulating the purified enveloped virus into a pharmaceutical formulation or into a solution suitable for infecting a cell.

The present disclosure also provides a purified enveloped virus produced by the method described herein.

### BRIEF DESCRIPTION OF THE DRAWINGS

FIG. 1 is a graphical representation showing (A) viable cell number and viability and (B) infectious titer yield of cells following 6 days in the production bioreactor using the acoustic wave method (black) for doxycycline removal compared with infectious titers from centrifugation (gray).

FIG. 2 is a graphical representation showing virus productivity by measuring (A) viable cell number and (B) infectious titer yield over time using the dilution method of doxycycline removal with different concentrations of doxycycline in the N−1 bioreactor.

FIG. 3 is a series of graphical representations showing (A) viable cell density and (B) cell viability in seed train bioreactor supplemented with 1 ng/mL doxycycline, and production bioreactors following doxycycline removal using the dilution method, acoustic wave method and centrifugation method. (C) Infectious titer formation over time, (D) total virus yield at harvest, (E) cell specific productivity and (F) glucose and (G) lactate concentration in production bioreactors following doxycycline removal using the dilution method, acoustic wave method and centrifugation method.

FIG. 4 is a graphical representation showing (A) infectious titer formation and cell specific productivity and (B) viable cell density and percent viability following extended production bioreactor fermentation.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, molecular biology, microbiology, virology).

Unless otherwise indicated, the conventional techniques of molecular biology, microbiology, virology, recombinant DNA technology, peptide synthesis in solution, solid phase peptide synthesis, and immunology utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The term “about”, unless stated to the contrary, refers to +/−20%, more for example+/−10%, of the designated value. For the avoidance of doubt, the term “about” followed by a designated value is to be interpreted as also encompassing the exact designated value itself (for example, “about 10” also encompasses 10 exactly).

As used herein the term “from” in the shall be taken to indicate that a specified integer may be obtained from a particular source albeit not necessarily directly from that source (i.e., includes recombinantly obtained).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “enveloped virus” refers to DNA and RNA viruses that have a viral envelope. Envelopes are typically derived from host cell membranes (e.g., phospholipids and proteins), but may include viral glycoproteins on the surface of the envelope. Enveloped viruses also comprise a “capsid”, which is a protein layer between the envelope and viral genome. In one example, the enveloped virus is a retrovirus. For example, the enveloped virus is a lentivirus, e.g., human immunodeficiency virus.

As used herein, the term “cell culture fluid” or “cell culture medium” will be understood to encompass the fluid or medium in which cells are grown for the purpose of producing an enveloped virus. The fluid or medium does not comprise the cells (e.g., the cells may have been removed, e.g., by centrifugation and/or removal of supernatant).

As used herein, the term “cell culture” or “suspension cell culture” will be understood to refer to the collective of the cell culture fluid or medium and the cultured cells.

The term “suspension” in reference to the cell lines will be understood to refer to single cells or small aggregates of cells that are free-floating in the cell culture medium. For example, such cells function and multiply in an agitated growth medium, thus forming a suspension.

The term “purify” or “purifying” or “purification” shall be taken to mean the removal, whether completely or partially, of at least one impurity present in the cell culture fluid, which thereby improves the level of purity of enveloped virus in solution.

The term “impurity” or “impurities” shall be taken to include one or more components in the cell culture fluid other than the enveloped virus. For example, impurities may include process related impurities such as host cell DNA, host cell proteins, and media components (e.g., fetal bovine serum).

### Production of Enveloped Viruses

Methods for the production of enveloped viruses will be apparent to the skilled artisan and/or described, for example, in Ansorge et al., (2010) Biochem. Eng. J. 48: 362-377; Schweizer and Merten (2010) Curr. Gene Ther. 10: 474-486; and Rodrigues et al., (2011) Viral Gene Therapy. Xu, InTech. Chapter 2: 15-40.

### Enveloped Viruses

In one example, the virus is a retrovirus, for example, a lentivirus. Exemplary retroviruses are from alpha retroviruses (such avian leukosis virus (ALV)), from beta retroviruses (such as mouse mammary tumor virus (MMTV)), from gamma retroviruses (such as murine leukemia virus (MLV)), from delta retroviruses (such as human T-lymphotropic virus (HTLV)), from epsilon retroviruses (such as Walleye dermal sarcoma virus (WDSV)), from spumavirus (such as human foamy virus (HFV) or simian foamy virus (SFV)), from primate lentiviruses such as the different types of human immunodeficiency viruses (HIV), the different types of simian immunodeficiency viruses (SIV), or from non-primate mammal lentiviruses such as the equine infectious anemia virus (EIAV), from the feline immunodeficiency virus (FIV), the caprine arthritis-encephalitis virus (CAEV), or the ovine visna-maedi virus (VMV).

In some examples, the enveloped virus, e.g., the retrovirus, is pseudotyped, i.e., it comprises an envelope glycoprotein derived from a virus different from the virus from which it is derived, a modified envelope glycoprotein or a chimeric envelope glycoprotein.

### Transgene Expression

In some examples, the enveloped virus comprises a transgene introduced into its genome. The transgene will depend on the specific use for which the enveloped viral vector is intended. Exemplary transgenes include a transgene coding for a therapeutic RNA (e.g. encoding an antisense complementary RNA of a target RNA or DNA sequence), a transgene encoding for a protein that is deficient or absent in a subject affected with a pathology, or a transgene used for vaccination with DNA, i.e. a transgene coding for a protein, the expression of which will induce vaccination of the recipient body against said protein. In some examples, the transgene encodes a protein or nucleic acid useful for treating a hemoglobinopathy, e.g., sickle cell disease or a thalassemia. In some examples, the transgene encodes a protein or nucleic acid useful for treating a primary immunodeficiency. In some examples, the transgene encodes a protein or nucleic acid useful for treating Wiskott-Aldrich Syndrome. In some examples, the transgene encodes a protein or nucleic acid useful for treating X linked agammaglobulinemia.

In some examples, an enveloped virus is produced by introducing the four following elements into a host cell: an expression cassette comprising a lentiviral gene gagpol, an expression cassette comprising a lentiviral gene rev, a transgene, all positioned between a lentiviral LTR-5′ and a lentiviral LTR-3′, and an expression cassette encoding envelope glycoprotein(s).

### Producer Cell Lines

In some examples, the enveloped virus is produced from a stable line expressing one or several elements required for producing an enveloped virus (Miller (2001) Curr. Protoc. Hum. Genet. Chapter 12: Unit 12.5.; Rodrigues et al. 2011, supra). In one example, the enveloped virus is produced from a mammal host cell transfected transiently with one or several plasmids coding for the elements required for producing the virus. According to an alternative example, the elements are introduced into the cell by means of multiple plasmids: one plasmid bearing an expression cassette comprising a lentiviral gagpol gene, one plasmid bearing an expression cassette comprising a lentiviral rev gene, one plasmid bearing an expression cassette encoding the envelope glycoprotein(s), one plasmid bearing an expression cassette comprising a tetracycline transactivator (tTA) gene, and/or one plasmid bearing an expression cassette comprising a lentiviral tat gene. A transfer plasmid comprising an expression cassette with the transgene, comprised between a lentiviral LTR-5′ and LTR-3′, can be introduced as a concatemer along with a helper plasmid with an antibiotic resistance cassette to confer resistance to the producer cells.

The host cell may be selected from any cell allowing production of an enveloped virus. According to one example, the cell is selected from a human cell (HEK293, HEK293T, HEK293FT, HEK293OX, Te671, HT1080, CEM), a musteli cell (NIH-3T3), a mustelidae cell (Mpf), a canid cell (D17), and derivatives thereof. According to one example, the cell is selected from CHO cells, BHK cells, MDCK cells, C3H 10T1/2 cells, FLY I, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, B-50 cells, 3T3 cells, NIH3T3 cells, HepG2 cells, Saos-2 cells, Huh7 cells, HeLa cells, W163 cells, 211 cells, and 211 A cells, and derivatives thereof.

According to one example, the cell is selected from the GPR, GPRG, GPRT, GPRGT, and GPRTG cell lines. In another example, the cell is selected from a cell line derived from any of the above cell lines.

In one example, the cell line is a suspension cell line selected from GPR, GPRG, GPRT, GPRGT, and GPRTG cell lines. For example, the suspension cell line is a cell line derived from any of the above cell lines. In one example, the suspension cell line is a cell line derived from a GPRG cell line. In another example, the suspension cell line is a cell line derived from a GPRGT cell line. In a further example, the suspension cell line is a cell line derived from a GPRTG cell line.

Methods of adapting an adherent cell line to grow in suspension will be apparent to the skilled person and/or described herein

In one example, the enveloped virus is produced from stable producer cells. Stable producer cells can be derived from packaging cell lines, including as any of the cell lines disclosed herein. In some embodiments the packaging cell lines are GPRG or GPRTG cell lines (Throm et al. (2009) Blood 113(21):5104-5110; and Bonner et al. (2015) Molecular Therapy, Vol. 23, Suppl. 1, S35). In one example, stable producer cell line cells are generated by synthesizing a vector by cloning one or more genes into a recombinant plasmid; forming a concatemeric array from an expression cassette excised from the synthesized vector, and an expression cassette obtained from an antibiotic resistance cassette plasmid; transfecting packaging cell line cells with the formed concatemeric array; and selecting and isolating the stable producer cell line cells. Virus is produced by inducing the inducible promoters of the stable producer cell line cells.

The present disclosure also provides a stable producer cell clone capable of producing an enveloped virus.

In one example, the stable producer cell clone is cultured to produce a stable producer cell line. Methods of culturing the stable producer cell clone to generate a stable producer cell line are described herein and/or are apparent to the skilled person.

The present disclosure also provides a stable producer cell clone capable of producing an enveloped virus with an infectious titer of at least 5×10⁶ TU/mL of culture medium and/or a viability of at least 75% and/or a viable cell density of at least 1.0×10⁷ cells/mL of culture medium for at least 15 days. For example, the stable producer cell clone is capable of producing an enveloped virus with an infectious titer of at least 5×10⁶ TU/mL of culture medium and/or a viability of at least 75% and/or a viable cell density of at least 1.0×10⁷ cells/mL of culture medium at day 15 of culture.

The present disclosure also provides a stable producer cell clone capable of producing an enveloped virus with an infectious titer of at least 5×10⁶ TU/mL of culture medium and/or a viability of at least 75% and/or a viable cell density of at least 1.0×10⁷ cells/mL of culture medium for at least 20 days. For example, the stable producer cell clone is capable of producing an enveloped virus with an infectious titer of at least 5×10⁶ TU/mL of culture medium and/or a viability of at least 75% and/or a viable cell density of at least 1.0×10⁷ cells/mL of culture medium at day 20 of culture.

The present disclosure also provides a stable producer cell clone capable of producing an enveloped virus with an infectious titer of at least 5×10⁶ TU/mL of culture medium and/or a viability of at least 75% and/or a viable cell density of at least 1.0×10⁷ cells/mL of culture medium for at least 25 days. For example, the stable producer cell clone is capable of producing an enveloped virus with an infectious titer of at least 5×10⁶ TU/mL of culture medium and/or a viability of at least 75% and/or a viable cell density of at least 1.0×10⁷ cells/mL of culture medium at day 25 of culture.

The present disclosure also provides a stable producer cell clone capable of producing an enveloped virus with an infectious titer of at least 5×10⁶ TU/mL of culture medium and/or a viability of at least 75% and/or a viable cell density of at least 1.0×10⁷ cells/mL of culture medium for at least 30 days. For example, the stable producer cell clone is capable of producing an enveloped virus with an infectious titer of at least 5×10⁶ TU/mL of culture medium and/or a viability of at least 75% and/or a viable cell density of at least 1.0×10⁷ cells/mL of culture medium at day 30 of culture.

The present disclosure also provides a stable producer cell clone capable of producing an enveloped virus with an infectious titer of at least 5×10⁶ TU/mL of culture medium and/or a viability of at least 75% and/or a viable cell density of at least 1.0×10⁷ cells/mL of culture medium for at least 35 days. For example, the stable producer cell clone is capable of producing an enveloped virus with an infectious titer of at least 5×10⁶ TU/mL of culture medium and/or a viability of at least 75% and/or a viable cell density of at least 1.0×10⁷ cells/mL of culture medium at day 35 of culture.

The present disclosure also provides a stable producer cell line capable of producing an enveloped virus with an infectious titer of at least 5×10⁶ TU/mL of culture medium and/or a viability of at least 75% and/or a viable cell density of at least 1.0×10⁷ cells/mL of culture medium for at least 15 days. For example, the stable producer cell line is capable of producing an enveloped virus with an infectious titer of at least 5×10⁶ TU/mL of culture medium and/or a viability of at least 75% and/or a viable cell density of at least 1.0×10⁷ cells/mL of culture medium at day 15 of culture.

The present disclosure also provides a stable producer cell line capable of producing an enveloped virus with an infectious titer of at least 5×10⁶ TU/mL of culture medium and/or a viability of at least 75% and/or a viable cell density of at least 1.0×10⁷ cells/mL of culture medium for at least 20 days. For example, the stable producer cell line is capable of producing an enveloped virus with an infectious titer of at least 5×10⁶ TU/mL of culture medium and/or a viability of at least 75% and/or a viable cell density of at least 1.0×10⁷ cells/mL of culture medium at day 20 of culture.

The present disclosure also provides a stable producer cell line capable of producing an enveloped virus with an infectious titer of at least 5×10⁶ TU/mL of culture medium and/or a viability of at least 75% and/or a viable cell density of at least 1.0×10⁷ cells/mL of culture medium for at least 25 days. For example, the stable producer cell line is capable of producing an enveloped virus with an infectious titer of at least 5×10⁶ TU/mL of culture medium and/or a viability of at least 75% and/or a viable cell density of at least 1.0×10⁷ cells/mL of culture medium at day 25 of culture.

The present disclosure also provides a stable producer cell line capable of producing an enveloped virus with an infectious titer of at least 5×10⁶ TU/mL of culture medium and/or a viability of at least 75% and/or a viable cell density of at least 1.0×10⁷ cells/mL of culture medium for at least 30 days. For example, the stable producer cell line is capable of producing an enveloped virus with an infectious titer of at least 5×10⁶ TU/mL of culture medium and/or a viability of at least 75% and/or a viable cell density of at least 1.0×10⁷ cells/mL of culture medium at day 30 of culture.

The present disclosure also provides a stable producer cell line capable of producing an enveloped virus with an infectious titer of at least 5×10⁶ TU/mL of culture medium and/or a viability of at least 75% and/or a viable cell density of at least 1.0×10⁷ cells/mL of culture medium for at least 35 days. For example, the stable producer cell line is capable of producing an enveloped virus with an infectious titer of at least 5×10⁶ TU/mL of culture medium and/or a viability of at least 75% and/or a viable cell density of at least 1.0×10⁷ cells/mL of culture medium at day 35 of culture.

Methods of determining cell viability will be apparent to the skilled person and/or are described herein. For example, the cell viability is determined using a hemocytometer after trypan blue staining. For example, cell viability is determined by trypan blue exclusion and determining the percentage of living cells to total cells in the sample.

Methods of measuring infectious titer will be apparent to the skilled person and/or described herein. In one example, infectious titer is determined by transduction with a GFP LV followed by flow cytometry. For example, the method comprises incubating virus containing supernatants with HEK293T cells seeded on plates, followed by trypsinization and washing, and using flow cytometry to determine the percentage of GFP positive cells and calculating the infectious titer in transducing units (TU)/mL of media.

Methods of determining viable cell density will be apparent to the skilled person and/or are described herein. In one example, viable cell density is determined using a hemocytometer after trypan blue staining. For example, viable cell density is determined by trypan blue exclusion and determining the total number of living cells per mL of sample.

In one example, the stable producer cell clone and/or stable producer cell line comprises a feature of any cell line described herein.

In one example, the stable producer cell clone and/or the stable producer cell line expresses a tetracycline-suppressible gene expression system in a cell culture medium.

In one example, the stable producer cell clone and/or the stable producer cell line is adapted to grow in suspension cell culture.

In one example, the stable producer cell clone and/or stable producer cell line is selected from or derived from a GPR, a GPRG, a GPRT, a GPRGT or a GPRTG cell line.

The present disclosure also provides methods for producing a stable producer cell clone capable of producing an enveloped virus in a suspension cell culture, the method comprising culturing the stable producer cell clone for at least 15 days in a suspension cell culture, detecting cell viability and/or infectious titer yield and/or viable cell density and selecting a stable producer cell clone, wherein the stable producer cell clone is produced if one or more or all of the following criteria is met after at least 15 days of culture:
(i) an infectious titer of at least 5×10⁶ TU/mL of culture medium;
(ii) a viability of at least 75%; and
(iii) a viable cell density of at least 1.0×10⁷ cells/mL of culture medium.

The present disclosure also provides methods for producing a stable producer cell clone capable of producing an enveloped virus in a suspension cell culture, the method comprising culturing the stable producer cell clone for at least 20 days in a suspension cell culture, detecting cell viability and/or infectious titer yield and/or viable cell density and selecting a stable producer cell clone, wherein the stable producer cell clone is produced if one or more or all of the following criteria is met after at least 20 days of culture:
(i) an infectious titer of at least 5×10⁶ TU/mL of culture medium;
(ii) a viability of at least 75%; and
(iii) a viable cell density of at least 1.0×10⁷ cells/mL of culture medium.

The present disclosure also provides methods for producing a stable producer cell clone capable of producing an enveloped virus in a suspension cell culture, the method comprising culturing the stable producer cell clone for at least 25 days in a suspension cell culture, detecting cell viability and/or infectious titer yield and/or viable cell density and selecting a stable producer cell clone, wherein the stable producer cell clone is produced if one or more or all of the following criteria is met after at least 25 days of culture:
(i) an infectious titer of at least 5×10⁶ TU/mL of culture medium;
(ii) a viability of at least 75%; and
(iii) a viable cell density of at least 1.0×10⁷ cells/mL of culture medium.

The present disclosure also provides methods for producing a stable producer cell clone capable of producing an enveloped virus in a suspension cell culture, the method comprising culturing the stable producer cell clone for at least 30 days in a suspension cell culture, detecting cell viability and/or infectious titer yield and/or viable cell density and selecting a stable producer cell clone, wherein the stable producer cell clone is produced if one or more or all of the following criteria is met after at least 30 days of culture:
(i) an infectious titer of at least 5×10⁶ TU/mL of culture medium;
(ii) a viability of at least 75%; and
(iii) a viable cell density of at least 1.0×10⁷ cells/mL of culture medium.

The present disclosure also provides methods for producing a stable producer cell clone capable of producing an enveloped virus in a suspension cell culture, the method comprising culturing the stable producer cell clone for at least 35 days in a suspension cell culture, detecting cell viability and/or infectious titer yield and/or viable cell density and selecting a stable producer cell clone, wherein the stable producer cell clone is produced if one or more or all of the following criteria is met after at least 35 days of culture:
(i) an infectious titer of at least 5×10⁶ TU/mL of culture medium;
(ii) a viability of at least 75%; and
(iii) a viable cell density of at least 1.0×10⁷ cells/mL of culture medium.

### Cell Culture Medium

The cells are cultivated in a medium suitable for cultivation of mammal cells and for producing an enveloped virus. The cells can be cultivated in an adherent environment, e.g., while attached to a surface, or in a suspension environment, e.g., suspended in the medium. The medium may moreover be supplemented with additives known in the field such as antibiotics, serum (notably fetal calf serum, etc.) added in suitable concentrations. The medium may be supplemented with GlutaMax™, Pluronic™ F-68 (ThermoFisher), LONG® R3 IGF-I (Sigma-Aldrich), Cell Boost™ 5, and/or an anticlumping agent. The medium used may notably comprise serum or be serum-free. Culture media for mammal cells are known and include, for example, DMEM (Dulbecco's Modified Eagle's medium) medium, RPMI1640 or a mixture of various culture media, including for example DMEM/F12, or a serum-free medium like optiMEM®, optiPRO®, optiPRO-SFM®, CD293® (ThermoFisher), TransFx™ (Cytiva), BalanCD® (Irvine), Freestyle F17® (Life Technologies), or Ex-Cell® 293 (Sigma-Aldrich).

In one example, the cells are cultivated in a culture media comprising TransFx™ (Cytiva).

In one example, the cells are supplemented with one or more additives selected from the group consisting of GlutaMax™, Cell Boost™ 5, poloxamer 188 and combinations thereof. For example, the cells are supplemented with GlutaMax™. In a further example, the cells are supplemented with Cell Boost™ 5. In one example, the cells are supplemented with poloxamer 188. In another example, the cells are supplemented with GlutaMax™ and Cell Boost™ 5. In a further example, the cells are supplemented with GlutaMax™ and poloxamer 188. In another example, the cells are supplemented with Cell Boost™ 5 and poloxamer 188. In one example, the cells are supplemented with GlutaMax™, Cell Boost™ 5 and poloxamer 188.

In one example, cells are supplemented with less than 15 mM GlutaMax™. For example, the cells are supplemented with about 15 mM GlutaMax™, or about 14 mM GlutaMax™, or about 13 mM GlutaMax™, or about 12 mM GlutaMax™, or about 11 mM GlutaMax™, or about 10 mM GlutaMax™. In one example, the cells are supplemented with less than 10 mM GlutaMax™. For example, the cells are supplemented with about 10 mM GlutaMax™, or about 9 mM GlutaMax™, or about 8 mM GlutaMax™, or about 7 mM GlutaMax™, or about 6 mM GlutaMax™, or about 5 mM GlutaMax™. In one example, cells are supplemented with less than 5 mM GlutaMax™. For example, the cells are supplemented with about 5 mM GlutaMax™, or about 4 mM GlutaMax™, or about 3 mM GlutaMax™, or about 2 mM GlutaMax™, or about 1 mM GlutaMax™. In one example, the cells are supplemented with between 1 mM and 10 mM GlutaMax™. For example, the cells are supplement with between 4 mM and 8 mM GlutaMax™. In one example, the cells are supplemented with 4 mM GlutaMax™. In another example, the cells are supplemented with 5 mM GlutaMax™. In a further example, the cells are supplemented with 6 mM GlutaMax™. In one example, the cells are supplemented with 7 mM GlutaMax™. In a further example, the cells are supplemented with 8 mM GlutaMax™.

In one example, the cells are supplemented with between 0.05% and 1% poloxamer 188. For example, the cells are supplemented with between 0.05 and 0.5% poloxamer 188. In one example, the cells are supplemented with between 0.08% and 0.2% poloxamer 188. For example, the cells are supplemented with 0.08% poloxamer 188. In another example, the cells are supplemented with 0.09% poloxamer 188. In a further example, the cells are supplemented with 0.1% poloxamer 188. In one example, the cells are supplemented with 0.15% poloxamer 188. In a further example, the cells are supplemented with 0.2% poloxamer 188.

In one example, the cells are supplemented with less than 10% Cell Boost™ 5. For example, the cells are supplemented with about 10% Cell Boost™ 5, or about 9% Cell Boost™ 5, or about 8% Cell Boost™ 5, or about 7% Cell Boost™ 5, or about 6% Cell Boost™ 5. In one example, the cells are supplemented with less than 5% Cell Boost™ 5. For example, the cells are supplemented with about 5% Cell Boost™ 5, or about 4% Cell Boost™ 5, or about 3% Cell Boost™ 5, or about 2% Cell Boost™ 5, or about 1% Cell Boost™ 5. In one example, the cells are supplemented with between 1% and 10% Cell Boost™ 5. For example, the cells are supplemented with between 2% and 8% Cell Boost™ 5. In one example, the cells are supplemented with between 4% and 6% Cell Boost™ 5. For example, the cells are supplemented with about 4% Cell Boost™ 5. In another example, the cells are supplemented with about 5% Cell Boost™ 5. In a further example, the cells are supplemented with about 6% Cell Boost™ 5.

In a process using transiently transfected cells, any agent allowing transfection of plasmids may be used. Exemplary agents include calcium phosphate or polyethyleneimine. The conditions (e.g., amount of plasmid(s), ratio between the plasmids, ratio between the plasmid(s) and the transfection agent, the type of medium, etc.) and the transfection time may be adapted by one skilled in the art according to the characteristics of the produced virus and/or of the transgene introduced into the transfer plasmid.

According to some examples, the culture medium used has a neutral pH (e.g. comprised between 7 and 7.4, notably 7, 7.1, 7.2, 7.3 or 7.4) conventionally used in the state of the art for cultivating cells and producing viruses. In one example, the suspension cell culture is at a pH of between 6.0 and 8.0. For example, the pH of the culture medium is 7.1±0.15. In other examples, the production process used comprises the cultivation of producing cells in a moderately acid medium. The expression “moderately acid condition” designates the pH of an aqueous solution comprised between 5 and 6.8, for example between 5.5 and 6.5, such as between 5.8 and 6.2. The selected pH will also depend on the buffering power of the culture medium used, which one skilled in the art may easily determine taking into account his/her general knowledge. One skilled in the art is able to modify the pH of a solution.

In one example, the production of the enveloped virus comprises: transient transfection of HEK293T cells or derivatives thereof by means of one or several plasmids coding for the elements required for production of said enveloped vector, or by the use of stable producing cells, e.g., GPRG or GPRTG stably transfected with a gene of interest, producing the vectors constitutively or after induction; culturing the cells in a suitable medium, for which the pH is of about 6 or of about 7; harvesting cell culture medium containing the enveloped virus.

### Suspension Cell Culture

The present disclosure provides a method of producing an enveloped virus in a suspension cell culture. For example, the method comprises culturing a suspension cell line expressing a tetracycline-suppressible gene expression system in a cell culture medium.

Methods of the disclosure are applicable to producing enveloped viruses from both small- and large-scale productions. The methods are particularly useful for their ability to be scaled up for manufacturing pharmaceutical products at commercial scale.

It will be apparent to the skilled person that production of an enveloped virus includes a cell expansion phase and a viral production phase.

The skilled person will understand that the cell expansion phase includes a seed train cell culture. As used herein, the term “seed train” refers to the generation of an adequate number of cells (i.e., cell growth) for viral production. The skilled person will understand that seed train cell culture comprises several cultivation systems which become larger with each passage (e.g. T-flasks, roller bottles or shake flasks, small scale bioreactor systems and subsequently larger bioreactors) in order to scale the culture from a small volume of cells to a larger volume of cells suitable for virus production.

It will be apparent to the skilled person that in any method described herein, that during the cell expansion phase the cells are cultured in the presence of tetracycline or a derivative thereof to suppress virus production but permit cell growth.

In one example, the cells are grown in a cell expansion phase prior to virus production.

In one example, the cell expansion phase is carried out in an expansion bioreactor (also termed an N−1 bioreactor).

In one example, the viral production phase is carried out in a production bioreactor (also termed an N bioreactor).

In one example, the cell expansion phase and viral production phase are carried out in the same vessel. For example, expansion of the suspension cell line and production of the enveloped virus occur in the same vessel. For example, the cell expansion phase and viral production phase are carried out in the same bioreactor.

In one example, the cell expansion phase and viral production phase are carried out in different vessels. For example, the cell expansion phase is carried out in an expansion bioreactor and viral production phase is carried out in a production bioreactor, wherein the expansion bioreactor and the production bioreactor are different.

In one example, the cell culture is operated in a batch, fed batch, continuous, semi-continuous, or perfusion mode.

In one example, the cell expansion phase and/or viral production phase are operated in a batch, fed batch, continuous, semi-continuous, or perfusion mode.

In one example, the cell expansion phase is carried out in batch, fed batch, continuous, semi-continuous, or perfusion mode. In one example, the cell expansion phase is carried out in batch mode. In another example, the cell expansion phase is carried out in fed-batch mode. In a further example, the cell expansion phase is carried out in continuous mode. In one example, the cell expansion phase is carried out in perfusion mode. In another example, the cell expansion phase is carried out in batch and perfusion mode. For example, the cell expansion phase is initially carried out in batch mode and subsequently carried out in perfusion mode.

In one example, the viral production phase is carried out in batch, fed batch, continuous, semi-continuous, or perfusion mode. In one example, the viral production phase is carried out in batch mode. In another example, the viral production phase is carried out in fed-batch mode. In a further example, the viral production phase is carried out in continuous mode. In one example, the viral production phase is carried out in perfusion mode. In another example, the viral production phase is carried out in batch and perfusion mode. For example, the viral production phase is initially carried out in batch mode and subsequently carried out in perfusion mode.

In one example, the cell expansion and the viral production phases are carried out in batch mode. In another example, the cell expansion and the viral production phases are carried out in perfusion mode. In a further example, the cell expansion phase is carried out in batch mode and the viral production phase is carried out in perfusion mode.

It will be apparent to the skilled person that reference to a batch, fed-batch, continuous and/or perfusion mode for a particular phase of cell culture (i.e., cell expansion and/or viral production) does not mean that the entire culture phase is carried out in that mode. For example, it only means that a period of the cell culture phase (e.g., at least 1 day) is carried out in that mode. It will also be understood that the mode does not necessary commence on day 0 of the culture phase. For example, the culture may commenced on day 0 and perfusion mode only commenced on day 2 of the cell culture phase.

In one example, the suspension cell culture is operated in batch mode. It will be apparent to the skilled person that “batch mode” refers to a process where cells are initially cultured in a medium and this medium is neither removed, replaced, nor supplemented, i.e., the cells are not “fed” with new medium, during or before the end of cultivation.

In one example, the suspension cell culture is operated in fed-batch mode. It will be apparent to the skilled person that “fed-batch mode” refers to a process where one or more nutrients are fed to the bioreactor during the cultivation period. In one example, the cell expansion phase and/or the virus production phase are operated in fed-batch mode. In one example, the cell expansion phase is operated in fed-batch mode.

In one example, the suspension cell culture is operated in perfusion mode. It will apparent to the skilled person that “perfusion mode” involves the constant feeding of fresh media and removal of spent media while retaining high numbers of viable cells (i.e., continuous media exchange). In one example, the cell expansion phase and/or the virus production phase are operated in perfusion mode. In one example, the virus production phase is operated in perfusion mode. In one example, perfusion mode involves perfusing with tetracycline or derivative-containing media throughout the cell expansion phase. In one example, perfusion mode involves initially perfusing with tetracycline or derivative-containing media, followed by perfusion with tetracycline or derivative-free media prior to induction.

In one example, the cells are cultured in fluidized bed bioreactors, hollow fiber bioreactors, roller bottles, shake flasks, or stirred tank bioreactors. In one example, cells are cultured in a stirred tank bioreactor. In examples, the cells are cultured in a Biostat® or Univessel® bioreactor (Sartorius).

In one example, the volume of the cell culture can be for example, about 0.01 L to about 0.1 L, or about 0.1 L to about 1 L, or about 1 L to about 5 L. In another example, the volume of the cell culture can be about 5 L to about 10 L, about 10 L to about 50 L, about 50 L to about 100 L, about 100 L to about 200 L, about 200 L to about 500 L, about 500 L to about 1000 L, about 1000 L to about 2000 L, or about 2000 L to about 5000 L. In one example, the volume of the cell culture is between about 35 and 150 L. In one example, the volume of the cell culture is about 35-150 L. In one example, the volume of the cell culture is about 50-70 L.

In one example, the suspension cell culture is operated at a temperature that permits cell growth and viral production. For example, the cell culture has a temperature conventionally used in the state of the art for cultivating cells and producing viruses. In one example, the suspension cell culture is at a temperature of between 35-39° C. For example, at a temperature of 37±0.5° C. or at a temperature of 38±0.5° C.

In one example, the suspension cell culture is operated at large-scale. For example, the suspension cell culture is operated at commercial-scale.

In one example, the suspension cell culture is operated for a period of at least 10 days. For example, the suspension cell cultures is operated for a period of between about 10 and 50 days. In one example, the suspension cell culture is operated for a period of between 10 and 35 days, for example, about 10 days or about 12 days, or about 15 days, or about 18 days, or about 20 days, or about 22 days, or about 25 days, or about 28 days, or about 30 days or about 32 days or about 35 days. In one example, the suspension cell culture is operated for at least 15 days. For example, the suspension cell culture is operated for about 20 days. In another example, the suspension cell culture is operated for at least 20 days. In a further example, the suspension cell culture is operated for at least 25 days. For example, the suspension cell culture is operated for about 28 days. In one example, the suspension cell culture is operated for at least 30 days. In one example, the suspension cell culture is operated for at least 32 days. For example, the suspension cell culture is operated for a period of 35 days. In one example, the suspension cell culture is operated for at least 35 days.

### Reducing Tetracycline and Derivatives Thereof in the Suspension Cell Culture

Methods of the disclosure are applicable to producing enveloped viruses from both small- and large-scale productions. The methods are particularly useful for their ability to be scaled up for manufacturing pharmaceutical products at commercial scale.

The present disclosure provides methods for improving the production of enveloped virus from a suspension cell culture. In particular, the present disclosure provides methods for removing or reducing the concentration of tetracycline or derivatives thereof from a suspension cell culture for production of an enveloped virus, wherein the cell line expresses a tetracycline-suppressible gene expression system. It will be apparent to the skilled person from the disclosure herein, that the methods of the disclosure result in increased cell quality and virus production.

It will be apparent to the skilled person from the disclosure herein that the concentration of tetracycline or derivative thereof need not be completely removed or reduced. For example, the concentration of tetracycline or derivative thereof is reduced by at least 50%, or at least 60%, or at least 70%, or at least 80%, or at least 85%, or at least 90%, or at least 95%.

As used herein, reference to a cell line expressing a “tetracycline-suppressible gene expression system” or “Tet-OFF” system refers to cell line that stably expresses tetracycline-controlled transactivator (tTA) such that the presence of tetracycline or a derivative thereof (e.g., doxycycline) silences transcription from tetracycline responsive element promoters.

The skilled person will understand that initially cells are cultured in the presence of tetracycline or a derivative thereof to suppress virus production but allow cell growth or expansion of the cell line.

In one example, the amount of tetracycline or a derivative thereof in the cell culture medium required to suppress virus production is at least 0.1 ng/mL.

In one example, the tetracycline or a derivative thereof in the cell culture medium suppresses virus production for between 1 and 20 days. For example, the amount of tetracycline or a derivative thereof in the cell culture medium suppresses virus production for about 1 day, or about 2 days, or about 3 days, or about 4 days, or about 5 days, or about 6 days, or about 7 days, or about 8 days, or about 9 days, or about 10 days. In another example, the amount of tetracycline or a derivative thereof in the cell culture medium suppresses virus production for about 11 days, or about 12 days, or about 13 days, or about 14 days, or about 15 days, or about 16 days, or about 17 days, or about 18 days, or about 19 days, or about 20 days.

It will be apparent to the skilled person from the disclosure herein that the tetracycline or a derivative thereof is added to the cell culture medium as a single bolus feed, as multiple feeds, or continuously over the duration of the culture. In one example, the tetracycline or a derivative thereof is added to the cell culture medium as a single bolus feed. For example, the tetracycline or a derivative thereof is added to the cell culture medium at the start of the cell culture (i.e., Day 0). In another example, the tetracycline or a derivative thereof is added to the cell culture medium every day or every second day for the duration of the cell culture. In one example, the tetracycline or a derivative thereof is added to the cell culture medium by perfusion cell culture at a defined concentration. For example, the tetracycline or a derivative thereof is added to the cell culture medium via perfusion cell culture at a concentration of 1.5 ng/mL. In a further example, the tetracycline or a derivative thereof is added to the cell culture medium as required to maintain a minimum concentration of tetracycline or a derivative thereof in the cell culture medium. For example, tetracycline or a derivative thereof is added to the cell culture medium to maintain a concentration of at least 0.1 ng/mL in the cell culture medium.

It will be apparent to the skilled person that withdrawal (or a reduction in the concentration) of tetracycline or a derivative thereof results in expression of the enveloped virus. For example, the concentration of tetracycline or derivative thereof in the cell culture medium is reduced to a concentration of 0.5 ng/mL or less.

The inventors have found that during the growth phase of the cell culture as cell density increases, the concentration of tetracycline or equivalent required to suppress virus production also increases. The inventors have found that without replenishing the tetracycline or equivalent in the cell culture, viral production is induced over time.

As discussed herein, typical methods of removing tetracycline or derivatives thereof from suspension cell cultures includes washing cells with a tetracycline or derivative-free cell culture medium by centrifuging cells, removing supernatant and subsequently resuspending cells. However, applying this method to suspension cell lines results in high shear forces on the cells resulting in lowering of cell quality and increased risks of contamination due to manual steps. In addition, centrifugation cannot be readily scaled up to commercial scale as these typical methods involve manual handling steps and are time consuming which can add several hours of process time.

The inventors' solution to these problems is to either dilute the suspension cell culture with a tetracycline or derivative-free cell culture medium; or retain the suspension cell line cells using an acoustic standing wave, remove a portion of the tetracycline or derivative-containing cell culture medium from the suspension cell culture, and contact the retained suspension cell line cells with a tetracycline or derivative-free cell culture medium. Both these methods improved cell quality with less stress on cells and fewer manual handling steps compared to traditional centrifugation methods. The inventors' solutions also reduce the risk of contamination of the culture by utilisation of a closed system. The methods of the disclosure also resulted in increased viral infectious titer yield.

Accordingly, the present disclosure provides methods for removing tetracycline or derivatives thereof from a suspension cell culture for production of an enveloped virus, wherein the method comprises: (i) diluting the suspension cell culture with a tetracycline or derivative-free cell culture medium; or (ii) retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

### Diluting the Suspension Cell Culture

In one example, the present disclosure provides a method for removing tetracycline or derivatives thereof from a suspension cell culture for production of an enveloped virus, comprising diluting the suspension cell culture with a tetracycline or derivative-free cell culture medium.

As used herein, the term “diluting” or “dilute” in reference to the cell culture will be understood to mean decreasing the concentration of a solute (i.e., tetracycline or derivative thereof) in the cell culture.

As discussed herein, the inventors' determined that it is not necessary to completely remove the tetracycline or derivative thereof from the cell culture to induce viral production. That is, the concentration of tetracycline or derivative thereof needed only to be reduced rather than being completely eliminated. This was an unexpected result because common protocols for inducing Tet-OFF cells call for removal of all or substantially all of the tetracycline or derivative thereof, for example through one or more wash steps. It was unexpected that merely diluting the spent tetracycline or derivative-containing media, rather than removing it, would be effective for inducing viral production.

For example, the concentration of tetracycline or derivative thereof needed only to be reduced to a concentration of less than about 0.1 ng/mL. Accordingly, the inventors' determined that a tetracycline or derivative thereof concentration of less than about 0.1 ng/mL permitted virus production. In another example, the concentration of tetracycline or derivative thereof needed only to be reduced to a concentration of less than about 0.2 ng/mL. Accordingly, the inventors' determined that a tetracycline or derivative thereof concentration of less than about 0.2 ng/mL permitted virus production. In another example, the concentration of tetracycline or derivative thereof needed only to be reduced to a concentration of less than about 0.5 ng/mL. Accordingly, the inventors' determined that a tetracycline or derivative thereof concentration of less than about 0.5 ng/mL permitted virus production.

In one example, the concentration of tetracycline or derivative thereof in the cell culture is diluted by addition of tetracycline or derivative-free cell culture medium. For example, the tetracycline or derivative-free cell culture medium is added directly to the tetracycline or derivative-containing cell culture medium.

In one example, the tetracycline or derivative-free cell culture medium is added to the suspension cell culture. It will be apparent to the skilled person that in this embodiment of the disclosure that the cell expansion phase and viral production phase occur in the same vessel (i.e., bioreactor).

In one example, the suspension cell culture comprising the tetracycline or derivative-containing cell culture medium is added to the tetracycline or derivative-free cell culture medium. It will be apparent to the skilled person that in this embodiment of the disclosure that the cell expansion phase and the viral production phase occur in separate vessels (i.e., bioreactors). For example, the suspension cell culture comprising the tetracycline or derivative-containing cell culture medium is cultured in a first bioreactor and is added to the tetracycline or derivative-free cell culture medium in a second bioreactor. For example, the suspension cell culture is transferred from a first bioreactor comprising tetracycline or derivative-containing cell culture medium into a second bioreactor, wherein the second bioreactor comprises tetracycline or derivative-free cell culture medium.

It will be apparent to the skilled person from the disclosure that reference to a first and subsequent vessel (i.e. bioreactor) is not reference to a defined or specific bioreactor and is for the purposes of comparison only. The first, second (and any subsequent) vessel may be separated by any number of other vessels.

In one example, the suspension cell culture comprising the tetracycline or derivative-containing cell culture medium is cultured in an expansion bioreactor and the suspension cell culture comprising the tetracycline or derivative-containing cell culture medium is added to the tetracycline or derivative-free cell culture medium in a production bioreactor. For example, the suspension cell culture is transferred from an expansion bioreactor comprising the tetracycline or derivative-containing cell culture medium to a production bioreactor, wherein the production bioreactor comprises tetracycline or derivative-free cell culture medium. In one example, the production bioreactor has been filled with preconditioned tetracycline or derivative-free cell culture medium (of set temperature, pH, and dissolved oxygen levels), and the suspension cell culture is transferred from the expansion bioreactor into the preconditioned medium.

In one example, the suspension cell culture is diluted with the tetracycline or derivative-free cell culture medium at a ratio of between about 1:1 and 1:20. For example, the suspension cell culture is diluted with the tetracycline or derivative-free cell culture medium at a ratio of between 1:2 and 1:10, or a ratio of between 1:4 and 1:7. In one example, the suspension cell culture is diluted with the tetracycline or derivative-free cell culture medium at a ratio of between 1:2 and 1:10. For example, the suspension cell culture is diluted with the tetracycline or derivative-free cell culture medium at a ratio of about 1:2, or about 1:3, or about 1:4, or about 1:5, or about 1:6, or about 1:7, or about 1:8, or about 1:9, or about 1:10. In one example, the suspension cell culture is diluted with the tetracycline or derivative-free cell culture medium at a ratio of between 1:4 and 1:7. In one example, the suspension cell culture is diluted with the tetracycline or derivative-free cell culture medium at a ratio of about 1:4. In another example, the suspension cell culture is diluted with the tetracycline or derivative-free cell culture medium at a ratio of about 1:5. In a further example, the suspension cell culture is diluted with the tetracycline or derivative-free cell culture medium at a ratio of about 1:6. In one example, the suspension cell culture is diluted with the tetracycline or derivative-free cell culture medium at a ratio of about 1:7.

It will be apparent to the skilled person from the disclosure herein that the ratio at which the suspension cell culture is diluted with the tetracycline or derivative-free cell culture medium is dependent upon the concentration of tetracycline or derivative thereof in the suspension cell culture. For example, the skilled person will recognise that if the concentration of tetracycline or derivative thereof in the suspension cell culture is about 1 ng/mL, the suspension cell culture is diluted such that the concentration of tetracycline or derivative thereof in the cell culture medium is less than 0.1 ng/mL. In a further example, the skilled person will recognise that if the concentration of tetracycline or derivative thereof in the suspension cell culture is about 0.1 ng/mL, the suspension cell culture does not require dilution prior to virus production.

It will be apparent to the skilled person from the disclosure herein that the amount of tetracycline or derivative thereof at which virus production is induced is also dependent on the cell density. Higher density of cells require a higher concentration of tetracycline or derivative thereof to suppress induction.

### Acoustic Standing Wave Separation

In one example, the present disclosure provides a method for removing tetracycline or derivatives thereof from a suspension cell culture for production of an enveloped virus, the method comprising retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

Removing a portion of the tetracycline or derivative-containing cell culture medium from the suspension cell culture comprises removing at least about 10% of the tetracycline or derivative-containing cell culture medium. In one example, removing a portion of the tetracycline or derivative-containing cell culture medium from the suspension cell culture comprises removing all or substantially all of the tetracycline or derivative-containing cell culture medium. In one example, removing a portion of the tetracycline or derivative-containing cell culture medium from the suspension cell culture comprises removing about 10%, about 20%, about 30%, about 40%, about 50%, about 60%, about 70%, about 80%, about 90%, or about 100% of the tetracycline or derivative-containing cell culture medium.

In one example, after removing the portion of the tetracycline or derivative-containing cell culture medium from the suspension cell culture, the cell line cells remain in suspension. In another example, after removing the portion of the tetracycline or derivative-containing cell culture medium from the suspension cell culture, the cell line cells are not still in suspension and must be resuspended.

In one example, contacting the retained suspension cell line cells with a tetracycline or derivative-free cell culture medium comprises resuspending the retained suspension cell line cells in the tetracycline or derivative-free cell culture medium. In one example, contacting the retained suspension cell line cells with a tetracycline or derivative-free cell culture medium comprises flowing the suspension cell line cells into a vessel containing the tetracycline or derivative-free cell culture medium.

As discussed above, the inventors have shown that using an acoustic standing wave to separate the suspension cell line cells from the tetracycline or derivative-containing cell culture medium results in reduced cell stress compared to centrifugation methods, thus improving cell quality and virus titer production.

In one example, the method comprises the use of an acoustic chamber or acoustic wave device. For example, the method comprises (i) flowing the suspension cell culture comprising the tetracycline or derivative-containing cell culture medium through an acoustic standing wave within an acoustic chamber, (ii) retaining the suspension cell line cells within the acoustic chamber, and (iii) flowing a tetracycline or derivative-free cell culture medium through the acoustic chamber comprising the retained suspension cell line cells to contact the cell line cells with tetracycline or derivative-free cell culture medium.

Acoustic chambers or acoustic wave devices suitable for use in the present disclosure will be apparent to the skilled person and/or described herein. Exemplary acoustic wave devices employ ultrasonic particle separation technology as described in EP 0633049. Exemplary acoustic wave devices include devices as described in U.S. Pat. No. 10,773,194.

In one example, the method comprises (i) flowing the suspension cell culture comprising the tetracycline or derivative-containing cell culture medium from a first vessel through an acoustic standing wave within an acoustic chamber, (ii) retaining the suspension cell line cells within the acoustic chamber, (iii) flowing the tetracycline or derivative-free cell culture medium through the acoustic chamber comprising the retained suspension cell line cells to contact the cell line cells with the tetracycline or derivative-free cell culture medium, and (iv) flowing the suspension cell line cells and the tetracycline or derivative-free cell culture medium into a second vessel.

In one example, the method comprises (i) flowing the suspension cell culture comprising the tetracycline or derivative-containing cell culture medium from an expansion bioreactor through an acoustic standing wave within an acoustic chamber, (ii) retaining the suspension cell line cells within the acoustic chamber, and (iii) flowing the tetracycline or derivative-free cell culture medium through the acoustic chamber comprising the retained suspension cell line cells to contact the cell line cells with the tetracycline or derivative-free cell culture medium, and (iv) flowing the suspension cell line cells and the tetracycline or derivative-free cell culture medium into a production bioreactor.

It will be apparent to the skilled person that the use of an acoustic chamber as described herein facilitates in-line processing. Thus in some examples, the method is performed in-line.

As used herein, the term “in-line” in the context of a process step refers to a process step that is integrated into or combined with one or more other process steps, or that flows directly from or to another process step without requiring manual intervention or handling.

In one example, the acoustic standing wave is in-line between a first vessel and a second vessel.

In one example, the method comprises removing a portion of the suspension cell culture comprising the tetracycline or derivative-containing cell culture medium from a first vessel, retaining the suspension cell line cells from the portion of the suspension cell culture using an acoustic standing wave, and contacting the retained suspension cell line cells with a tetracycline or derivative-free cell culture medium in a second vessel.

In one example, the method comprises removing a portion of the suspension cell culture comprising the tetracycline or derivative-containing cell culture medium from an expansion bioreactor, retaining the suspension cell line cells from the portion of the suspension cell culture using an acoustic standing wave, and contacting the retained suspension cell line cells with a tetracycline or derivative-free cell culture medium in a production bioreactor.

### Purifying Enveloped Viruses

In one example, the enveloped virus is purified from the cell culture comprising one or more steps selected from the group consisting of clarification filtration, anion exchange chromatography, concentration and diafiltration.

The downstream process for purifying and concentrating viral vector from a cell culture includes a harvest filtration step (also known as “clarification filtration” or “harvest clarification filtration” or “bioburden reduction”) to remove cellular debris and components from the harvest, a purification step, e.g., anion exchange chromatography, to reduce overall volume and to separate viral vector from host cell DNA, proteins, and media components, and an ultrafiltration/diafiltration step to concentrate the viral vector into a final formulation buffer. In some examples, the downstream step further includes a sterile filtration step for removal of microorganisms from the final product.

As used herein, “harvesting” refers to removal of the cell culture media containing virus particles from the producer cells for downstream processing, and “harvest” refers to the cell culture media containing virus particles that has been removed for the purpose of downstream processing. A harvesting process may include collecting one or more harvests. “Harvest filtration” refers to either a harvest that has been filtered or cell culture media containing virus particles that has been filtered to remove the producer cells for downstream processing.

In one example, a harvested cell culture fluid is filtered following production of the enveloped virus.

As used herein, the term “filtered cell culture fluid” will be understood to encompass the cell culture fluid after it has been subjected to harvest filtration.

Following harvest filtration, the enveloped virus is purified using anion exchange.

In one example, the anion exchange is performed in bind-elute mode. In this regard, the enveloped virus binds to the anion exchanger while contaminants flow through. The virus is subsequently eluted from the anion exchanger. Performing anion exchange in this manner reduces the volume of liquid in which the virus is suspended and removes contaminants such as host cell DNA, host cell proteins, and medium components like fetal bovine serum.

Suitable anion exchangers will be apparent to the skilled artisan. Exemplary anion exchangers are a column comprising a resin or a membrane or another suitable substrate.

In one example, the anion exchanger is a weak anion exchanger, e.g., comprising an ion exchange group selected from a diethylaminoethyl (DEAE) or aminoethyl group.

In another example, the anion exchanger is a strong anion exchanger, e.g., comprising an ion exchange group selected from a quaternary ammonium (Q), diethyl-2-hydroxypropylaminoethyl (QAE), triethylaminoethyl (TEAE), or trimethyl aminoethyl group. Exemplary anion exchangers include MUSTANG® E, MUSTANG® Q, SARTOBIND® Q, CHROMASORB®, POSSIDYNE®, CAPTO® Q, QSFF, POROS® Q, FRACTOGEL® Q, NATRIX® Q.

In one example, the anion exchanger comprises a Q ion exchange group.

In one example, the anion exchanger is a membrane anion exchanger comprising a Q ion exchange group. For example, the anion exchanger is MUSTANG® Q.

In one example, an enveloped virus eluted from anion exchange column is further purified on the basis of its size. In one example, the buffer in which virus was eluted from the anion exchange column, is exchanged more or less at the same time. In the process of the disclosure, tangential flow filtration is preferred. This method permits impurity removal and buffer exchange at almost the same time.

Tangential flow ultrafiltration/diafiltration is a method which may be used to remove residual protein and nucleic acids as well as for exchanging working buffer into a final formulation buffer. Ultrafiltration using tangential flow is preferred and different devices can be used (e.g. Proflux and LABSCALE (ultrafiltration system) TFF System, both Millipore or the KR2i system from Repligen). The particular ultrafiltration membrane selected will be of a filter pore size sufficient small to retain enveloped virus but large enough to allow penetration of impurities. Depending on the manufacturer and membrane type, nominal molecular weight cut-offs between 100 and 1000 kDa may be appropriate (e.g. UFP-750-E-5A, GE Healthcare; BIOMAX (ultrafiltration device) NMWC 1000, Millipore). In one example, the molecular weight cut-off is 500 kDa. The membrane composition may be, but it is not limited to, regenerate cellulose, (modified) polyethersulfone, polysulfone. Membranes can be of flat sheet or hollow fibre type. The main parameters that must be optimized are flux rate and trans-membrane pressure. In combination with nominal molecular weight cut-off these two parameters will enable efficient purification and buffer exchange and high virus yield.

As an additional step sterile filtration may be performed to eliminate bioburden. Therefore diluted eluate or final retentate from the ultrafiltration step may be filtered through a filter, for example a 0.22 μm filter. The filter may be constructed from various materials, which may include but are not limited to polypropylene, hydrophilic PVDF, cellulose, hydrophilic regenerated cellulose, cellulose esters, wetting agent-free cellulose acetate, cellulose acetate, nylon, hydrophilic nylon membrane, polyethersulfone, hydrophilic polyethersulfone, hydrophilic asymmetric PES, or any other material which is consistent with low unspecific influenza virus binding. The filter may have a single membrane layer or more than one layer or may incorporate a prefilter of the same or different material, for example a 0.45 μm prefilter. The sterile filtrated virus can be held frozen for subsequent manipulation.

The present disclosure is described further in the following non-limiting examples.

### EXAMPLES

### Example 1: Adaption of Adherent Stable Packaging Cell Lines for Scalable Lentivirus Production

Stable producer pools were generated by stable concatemeric array transfections using a WASp-T2A-GFP construct and subsequent antibiotic selection. The parental cell line of all packaging and producer cell lines is the adherent HEK293T/17 cell line. The original adherent GPRG and GPRTG packaging cell lines were established by stable introduction of all genetic elements required for lentivirus production, except for the gene of interest (transfer gene).

The packaging cell lines GPRG and GPRTG were subsequently adapted to growth in suspension using different serum-free media. Briefly, each cell line was thawed from a master cell bank. Both cell lines are cultivated in adherent culture using 2.5 ng/mL doxycycline as well as 2 g/mL Puromycin. The cells were cultivated for two passages in D10 medium (DMEM+10% FBS) in static phase using T-Flasks. Cultivation in static phase involved culturing with a complete medium exchange every 48 to 96 h and keeping the cell density >1E6 cells/mL until the cells reached an appropriate doubling time (<50 h) and viability (>90%). A lower seeding density of 0.3-0.4E6 cells/mL was applied for seed expansion. After recovery of the cells a direct switch to serum-free medium was performed. Subsequent cultivation was performed in shaken environment at 120 rpm (19 mm orbit) using non-baffled shake flasks at a maximum working volume of 32% and 5-8% CO₂.

### Example 2: Establishment of Suspension Cell Culture

The packaging and producer cell lines used are based on an inducible TET-OFF expression system, where the presence of doxycycline inhibits the expression of lentiviral components, and the removal of doxycycline induces the production of lentiviruses.

Experiments were performed using the suspension-adapted CAL-H producer clone. Cells for the inoculation of the production bioreactor were expanded in an N−1 seedtrain bioreactor using doxycycline-supplemented media.

Briefly, seed expansion was achieved using the following conditions:

| Culture Day | Process step |
|---|---|
| Vial thaw | Vial thaw in 20 mL of media + 1.0 ng/mL dox. |
| | Total volume 20 mL (125 mL flask) |
| WD1 | WD1 top up with 20 mL of media + 1.0 ng/mL dox. |
| | Total volume 40 mL (125 mL flask) |
| | Passage every 48 hours (2 days) |
| WD4 | WD4 passage to 0.5 ± 0.1 × 10\hat{ }6 cells/mL |
| | In fresh media + 1.0 ng/mL dox |
| | Total volume 40 mL (125 mL flask) |
| WD6 | WD6 passage to 0.5 ± 0.1 × 10\hat{ }6 cells/mL |
| | In fresh media + 1.0 ng/mL dox |
| | Total volume 75 mL (250 mL flask) |
| WD8 | WD8 passage to 0.5 ± 0.1 × 10\hat{ }6 cells/mL |
| | In fresh media + 1.0 ng/mL dox |
| | Total volume 75 mL (250 mL flask) |
| WD10 | WD10 passage to 0.5 ± 0.1 × 10\hat{ }6 cells/mL |
| | In fresh media + 1.0 ng/mL dox |
| | Total volume 150 mL (500 mL flask) |
| WD12 | WD12 passage to 0.5 ± 0.1 × 10\hat{ }6 cells/mL |
| | In fresh media + 1.0 ng/mL dox |
| | Total volume 300 mL (1000 mL flask) |
| WD14 | WD14 passage (3 flasks) to 0.5 ± 0.1 × 10\hat{ }6 cells/mL |
| | In fresh media + 1.0 ng/mL dox |
| | Total volume 300 mL (1000 mL flask) |
| WD16 to | WD16 Inoculate N-1 Bioreactor |
| WD20 (N-1) | 1.0 ± 0.2 × 10\hat{ }6 cells/mL |
| | In fresh media + 1.0 ng/mL dox |
| | Total volume 4500 mL (5 L glass STR bioreactor) |

### Example 3: Removal of Doxycycline Using Acoustic Wave Method

Cells are washed with doxycycline-free medium using a device based on acoustic wave separation prior to inoculation of the production bioreactor to remove doxycycline from the cell culture medium.

An acoustic wave device was connected in-line between the N−1 bioreactor and the production bioreactor. Cells in doxycycline containing media flowed into the device and were retained in an acoustic wave field while media flowed out to a waste bag. Fresh doxycycline-free media was pumped in, and cells were released into the production bioreactor. The process was repeated until all cells were transferred into the production bioreactor in doxycycline-free cell culture medium.

Viable cell density and infectious titer of the samples was measured. Briefly, to determine infectious titer cells were sampled at various time points throughout the culture period and stained with antibody against human gamma-globin to determine the infectious titer of the sample measured in transducing units (TU)/mL.

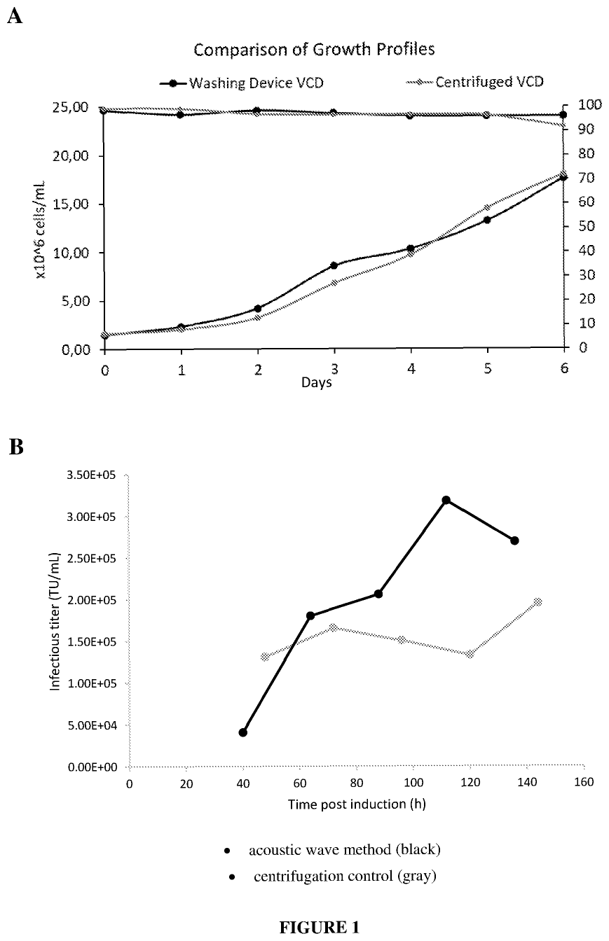

As shown in FIG. 1, removal of doxycycline using the acoustic wave method resulted in a viable cell density comparable to the typical centrifuge method. With the acoustic wave washing device, a daily cell bleed starting on day 4 was applied to keep the viable cell density comparable to the slower growing centrifuged culture. Titer yield was also comparable or superior to the centrifuge method.

### Example 4: Removal of Doxycycline Using Dilution Method

Cells were grown to about 5× higher density in the N−1 bioreactor to achieve a viable cell density of 6-10×10⁶ cells/mL and viability of ≥80%.

Cells were then directly transferred to the production bioreactor containing doxycycline-free media to achieve a target cell concentration of about 1.5-1.8×10⁶ cells/mL and 4.5 kg volume in the production bioreactor.

To investigate the effect of the concentration of doxycycline on virus production, cells were cultured in the N−1 bioreactor for 5 days (i.e., working days −5 to 0) in the presence of 0.1 ng/mL, 0.5 ng/mL, 1 ng/ml, 2 ng/mL, 2.5 ng/mL, or 5 ng/mL doxycycline. Note, doxycycline was not replenished throughout the N−1 culture.

However, a medium exchange was performed at working days −2 and −1 using media containing each respective doxycycline concentration.

On day 0, cells were directly transferred into the production bioreactor containing doxycycline-free media at a ratio of about 1:5. After transfer to the production bioreactor, to prevent stress to cells the bioreactor was run in batch mode for 2 days, followed by perfusion mode with fresh media.

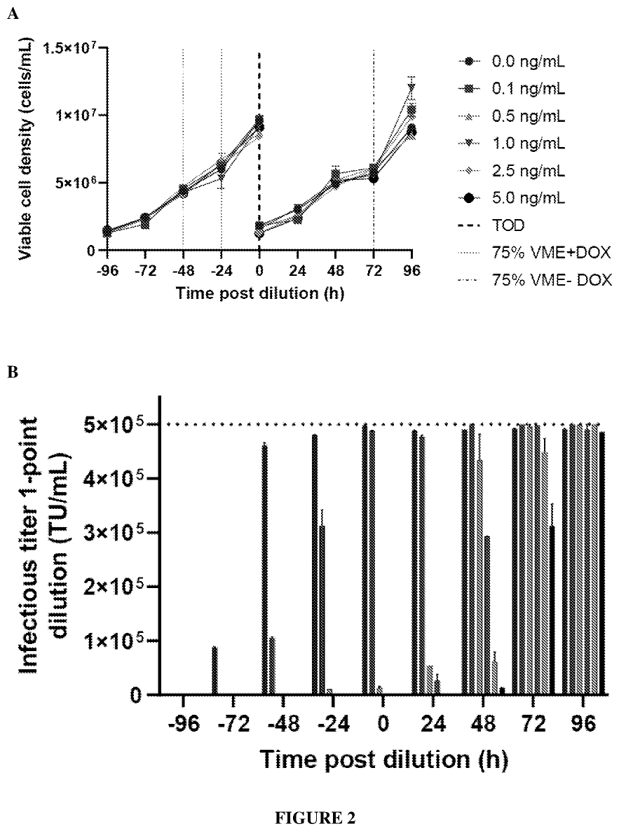

As shown in FIG. 2, a concentration of doxycycline as low as 0.1 ng/mL suppressed viral production for 2 days, whilst concentrations above 0.5 ng/mL suppressed viral production until at least day 1 in the production bioreactor. Cultures with >1.0 ng/mL doxycycline showed large increases in virus production around day 2 in the production bioreactor.

At the time of seeding the production bioreactor, a dilution of around 1:5 theoretically results in 0.2 ng/ml dox. Surprisingly, viral induction commenced prior to perfusion with fresh doxycycline-free media started and in the presence of low concentrations of doxycycline.

### Example 5: Comparison of Virus Induction Strategies

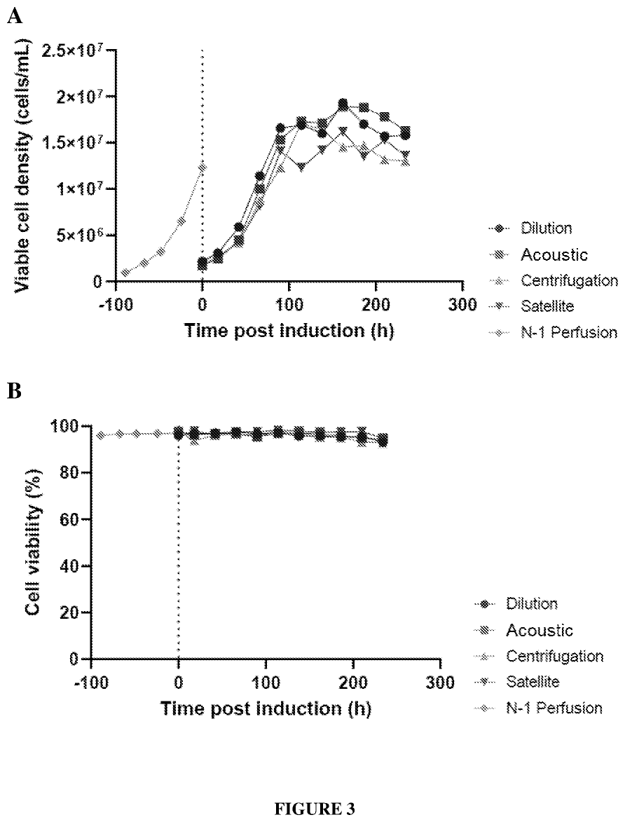

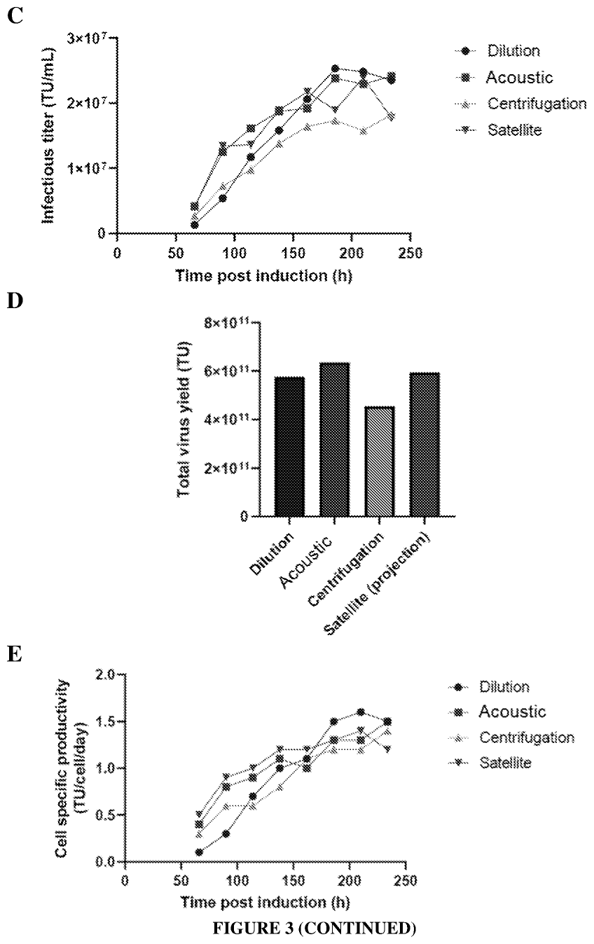

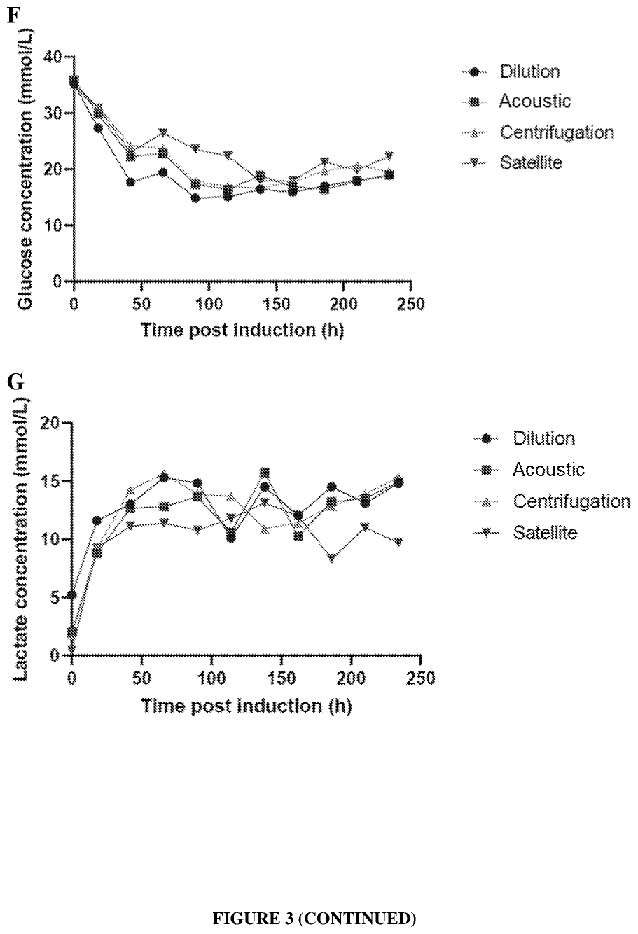

Initially cells were grown in a seedtrain bioreactor in the presence of 1 ng/mL doxycycline. After 65 hours of culture, perfusion with 1 ng/mL doxycycline was performed. As shown in FIG. 3A, high viable cell density and viability of cells was achieved.

A side-by-side comparison of the three different virus induction strategies (i.e., the dilution method, acoustic wave method and centrifugation) was performed.

The cells were grown to about 10-15×10⁶ cells/mL, after which a continuous cell bleed was performed at a fixed flow rate to maintain ˜15×10⁶ cells/mL cell density. As shown in FIG. 3B, a high cell viability was maintained through to day 10 in the production bioreactor.

Infectious titer was assessed daily from the bioreactors and as shown in FIGS. 3C and D, the 3 methods were comparable in terms of total virus yield, with centrifugation yield slightly lower. The other disadvantage of centrifugation is the exposure of cells to more shear forces.

As shown in FIG. 3E, cell specific productivity increased rapidly over time. pH was also measured over the duration and the pH remained with the deadband range such that no base addition was required (data not shown). As shown in FIGS. 3F and 3G, metabolite (i.e., lactate (FIG. 3G) and glucose (FIG. 3F)) concentrations were stable over time.

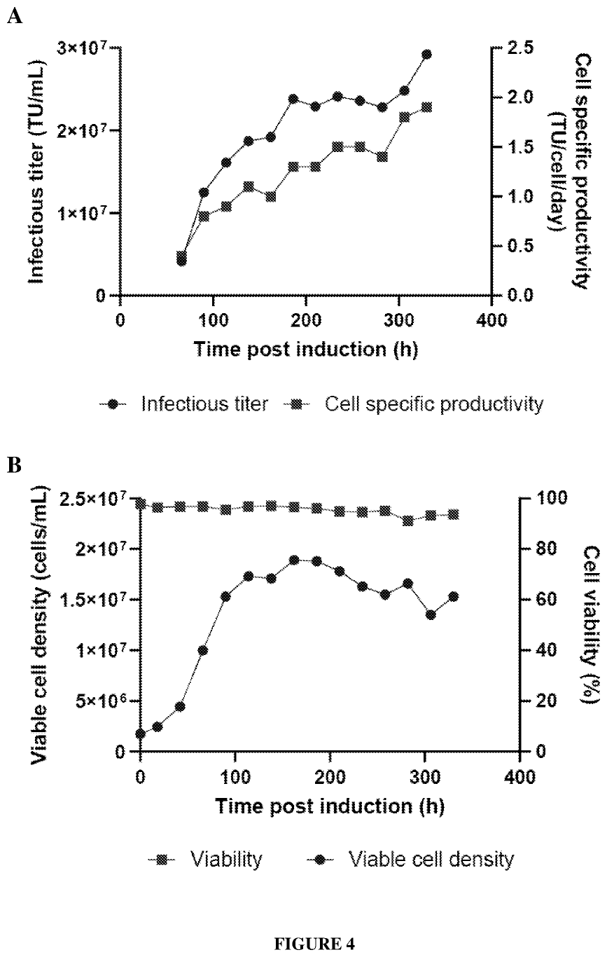

One production bioreactor run was extended beyond day 10 to day 12. As shown in FIG. 4, infectious titer and cell-specific productivity continued to increase after 12 days indicating that production could be extended. A cell bleed was applied to prevent overgrowth and maintain a cell density of about 1.5E7 cells/mL. The cell bleed strategy prevents nutrient depletion and prevents increase harvest turbidity which hampers the downstream process for purifying the virus.

### Example 6: Large-Scale Manufacturability Assessment

To assess scale-up and robustness of the suspension harvests, suspension cell lines GPRG and GPRTG were grown through to day 35 in the production bioreactor. Viable cell density, viability and infectious titer of stable producer cell clones was measured with high viable cell density, viability of cells and infectious titer achieved out to day 35.

These studies showed that the suspension stable producer cell lines were capable of producing enveloped virus at commercial scale (i.e., for at least a period of 15 days).

1. A method of producing an enveloped virus in a suspension cell culture, the method comprising culturing a suspension cell line expressing a tetracycline-suppressible gene expression system in a cell culture medium.

2. The method of claim 1, wherein the suspension cell line is a stable producer cell line.

3. The method of claim 1, wherein the suspension cell line is initially cultured in a cell culture medium comprising a sufficient amount of tetracycline or a derivative thereof to suppress production of the enveloped virus and allow expansion of the suspension cell line.

4. The method of claim 3, wherein the sufficient amount of tetracycline or a derivative thereof in the cell culture medium is at least 0.1 ng/mL.

5.-6. (canceled)

7. The method of claim 3, wherein the method comprises reducing the concentration of tetracycline or a derivative thereof in the cell culture medium such that production of the enveloped virus is induced.

8. The method of claim 7, wherein the concentration of tetracycline or derivative thereof in the cell culture medium is reduced to a concentration of 0.5 ng/mL or less.

9.-10. (canceled)

11. The method of claim 7, wherein the concentration of tetracycline or derivative thereof is reduced in the cell culture medium by:
(i) diluting the suspension cell culture with a tetracycline or derivative-free cell culture medium; or
(ii) retaining the suspension cell line cells using an acoustic standing wave, removing a portion of the tetracycline or derivative-containing cell culture medium from the suspension cell culture, and contacting the retained suspension cell line cells with a tetracycline or derivative-free cell culture medium.

12. The method of claim 11, wherein diluting the suspension cell culture comprises (i) adding the tetracycline or derivative-free cell culture medium directly to the tetracycline or derivative-containing cell culture medium, or (ii) adding tetracycline or derivative-free cell culture medium to the suspension cell culture.

13. (canceled)

14. The method of claim 11, wherein the suspension cell line is expanded and the enveloped virus is produced in the same vessel.

15. The method of claim 11, wherein the suspension cell line is grown to a viable cell density of between about 1×10⁵ cells/mL to about 1×10¹⁰ prior to diluting the suspension cell culture with the tetracycline or derivative-free cell culture medium.

16.-17. (canceled)

18. The method of claim 11, wherein the suspension cell culture is diluted with the tetracycline or derivative-free cell culture medium at a ratio of between about 1:1 and 1:20.

19. (canceled)

20. The method of claim 1, wherein the suspension cell line is initially seeded in the cell culture medium at a density of between about 1×10⁵ cells/mL and 1×10¹⁰ cells/mL.

21.-23. (canceled)

24. The method of claim 1, wherein the suspension cell culture is operated in perfusion mode.

25. The method of claim 1, wherein the suspension cell culture is operated for a period of at least 15 days.

26.-27. (canceled)

28. The method of claim 1, wherein the suspension cell culture has a volume of greater than about 1 L.

29. The method of claim 1, wherein the suspension cell culture is at a pH of between 6.0 and 8.0 and/or at a temperature of between 35-39° C.

30. The method of claim 1, wherein the method further comprises purifying the enveloped virus from the suspension cell culture.

31. The method of claim 30, wherein purifying the enveloped virus comprises one or more steps selected from the group consisting of clarification filtration, anion exchange chromatography, concentration and diafiltration.

32. The method of claim 30, additionally comprising formulating the purified enveloped virus into a pharmaceutical formulation or into a solution suitable for infecting a cell.

33. The method of claim 1, wherein the enveloped virus is a retrovirus.

34. (canceled)

35. (canceled)

36. A stable producer cell clone capable of producing an enveloped virus in a suspension cell culture with an infectious titer of at least 5×10⁶ TU/mL of culture medium and/or a viability of at least 75% and/or a viable cell density of at least 1.0×10⁷ cells/mL of culture medium for at least 15 days.

37. (canceled)

38. A method for producing a stable producer cell clone capable of producing an enveloped virus in a suspension cell culture, the method comprising culturing the stable producer cell clone for at least 15 days in a suspension cell culture, detecting cell viability and/or infectious titer yield and/or viable cell density and selecting a stable producer cell clone, wherein the stable producer cell clone is produced if one or more or all of the following criteria is met after at least 15 days of culture:
(i) an infectious titer of at least 5×10⁶ TU/mL of culture medium;
(ii) a viability of at least 75%; and
(iii) a viable cell density of at least 1.0×10⁷ cells/mL of culture medium.
